SEQUENCE LISTING (9 sequences)

SEQ ID NO: 1 - protein, 15 aa, synthetic construct
CASSPTSGGQETQYF

SEQ ID NO: 2 - protein, 25 aa, Severe acute respiratory syndrome-related coronavirus
MIELSLIDFYLCFLAFLLFLVLIML

SEQ ID NO: 3 - protein, 9 aa, Alphainfluenzavirus influenzae
GILGFVTFL

SEQ ID NO: 4 - protein, 9 aa, Flavivirga amylovorans
LLWNGPMAV

SEQ ID NO: 5 - protein, 10 aa, Severe acute respiratory syndrome-related coronavirus
LSPRWYFYYL

SEQ ID NO: 6 - protein, 12 aa, Severe acute respiratory syndrome-related coronavirus
VQELYSPIFLIV

SEQ ID NO: 7 - protein, 10 aa, Homo sapiens
GTSGSPIVNR

SEQ ID NO: 8 - protein, 9 aa, synthetic construct
CASSLNEQF

SEQ ID NO: 9 - protein, 15 aa, synthetic construct
CASSPTSGGQETQYF

(19) United States
(12) Patent Application Publication
(10) Pub. No.: US 2024/0339173 A1
(43) Pub. Date: Oct. 10, 2024

(54) SYSTEMS AND METHODS FOR A BIDIRECTIONAL LONG SHORT-TERM MEMORY EMBEDDING MODEL FOR T-CELL RECEPTOR ANALYSIS

(71) Applicants: Heewook Lee, Tempe, AZ (US); Pengfei Zhang, Tempe, AZ (US); Michael Cai, Scottsdale, AZ (US); Seojin Bang, Mountain View, CA (US)

(72) Inventors: Heewook Lee, Tempe, AZ (US); Pengfei Zhang, Tempe, AZ (US); Michael Cai, Scottsdale, AZ (US); Seojin Bang, Mountain View, CA (US)

(73) Assignee: Arizona Board of Regents on Behalf of Arizona State University, Tempe, AZ (US)

(21) Appl. No.: 18/631,922

(22) Filed: Apr. 10, 2024

Related U.S. Application Data
(60) Provisional application No. 63/458,236, filed on Apr. 10, 2023.

(51) Int. Cl. G16B 15/30; G06F 30/27; G16B 15/20; G16B 40/20

(52) U.S. Cl. CPC G16B 15/30; G06F 30/27; G16B 15/20; G16B 40/20

(57) ABSTRACT

A T-Cell receptor (TCR) specific embedding model uses a bidirectional long short-term memory (LSTM) to generate representations for TCR sequences and predict a “next token” in a TCR sequence. The embedding model can be trained in an unsupervised manner using a large collection of TCR sequences, and can be combined with downstream models to perform tasks, such as a TCR-epitope binding prediction model and a clustering algorithm. The embedding model demonstrates significant of prediction improvement when compared to existing models.

### CROSS-REFERENCE TO RELATED APPLICATIONS

This is a U.S. Non-Provisional Patent Application that claims benefit to U.S. Provisional Patent Application Ser. No. 63/458,236 filed 10 Apr. 2023, which is herein incorporated by reference in its entirety.

### SEQUENCE LISTING

The present application contains a Sequence Listing which has been submitted electronically in .XML format and is hereby incorporated herein by reference in its entirety. Said computer readable file, was created on 10 Apr. 2024 is named 055743_792759_SequenceListing.xml and is 9 kilobytes in size.

### FIELD

The present disclosure generally relates to T-Cell Receptor analysis, and in particular, to a computer-implemented system and associated methods for T-Cell Receptor analysis and its application to TCR-epitope binding prediction and clustering.

### BACKGROUND

T cell receptors (TCRs) play critical roles in adaptive immune systems as they enable T cells to distinguish abnormal cells from healthy cells. However, development of computational models to predict or otherwise characterize binding affinities for T cells and TCRs is a challenging task.

It is with these observations in mind, among others, that various aspects of the present disclosure were conceived and developed.

### SUMMARY

A system outlined herein includes a processor in communication with a memory, the memory including instructions executable by the processor to: apply a sequence of amino acid tokens as input to a character convolutional layer of an embedding model resulting in a plurality of convolutional latent vectors, each convolutional latent vector of the plurality of convolutional latent vectors being respectively associated with an amino acid token of the sequence of amino acid tokens; generate a plurality of token latent vectors from the plurality of convolutional latent vectors using a bidirectional long short-term memory (LSTM) stack of the embedding model, the bidirectional LSTM stack having a plurality of bidirectional LSTM layers that collectively model a joint probability of the sequence of amino acid tokens, each token latent vector of the plurality of token latent vectors representing an amino acid token of the sequence of amino acid tokens and encoding contextual relationships between amino acid tokens of the sequence of amino acid tokens; and combine the plurality of token latent vectors into a sequence representation vector for the sequence of amino acid tokens. In some examples, the embedding model has been trained using ground truth data including T cell receptor sequences.

The memory can further include instructions executable by the processor to: apply the sequence representation vector for the sequence of amino acid tokens as input to a downstream task element.

The bidirectional LSTM stack can include a forward pass sub-layer and a backward pass sub-layer for each respective bidirectional LSTM layer of the plurality of bidirectional LSTM layers. The forward pass sub-layer can have a set of forward layer weights and the backward pass sub-layer can have a set of backward layer weights that are jointly optimized during a training process of the embedding model, the set of forward layer weights and the set of backward layer weights being distinct from one another.

Each forward pass sub-layer can model a forward probability of a next right amino acid token of the sequence of amino acid tokens given one or more previous left tokens of the sequence of amino acid tokens. As such, the memory can further include instructions executable by the processor to: predict, at a softmax layer of the embedding model and based on an output of a forward pass sub-layer of a final bidirectional LSTM layer of the plurality of bidirectional LSTM layers, a next right amino acid token given one or more previous left tokens of the sequence of amino acid tokens.

Similarly, each backward pass sub-layer can model a backward probability of a next left amino acid token of the sequence of amino acid tokens given one or more previous right tokens of the sequence of amino acid tokens. As such, the memory can further include instructions executable by the processor to: predict, at a softmax layer of the embedding model and based on an output of a backward pass sub-layer of a final bidirectional LSTM layer of the plurality of bidirectional LSTM layers, a next left amino acid token given one or more previous right tokens of the sequence of amino acid tokens.

Each bidirectional LSTM layer of the plurality of bidirectional LSTM layers can respectively output a LSTM latent vector of a plurality of LSTM latent vectors associated with the amino acid token. The memory can further include instructions executable by the processor to: combine a convolutional latent vector associated with the amino acid token and the plurality of LSTM latent vectors associated with the amino acid token into a token latent vector of the plurality of token latent vectors for the amino acid token.

In some examples, the sequence representation vector for the sequence of amino acid tokens can be an element-wise average of the plurality of token latent vectors.

The character convolutional layer can include a plurality of convolutional layers, each convolutional layer of the plurality of convolutional layers being followed by a maxpooling layer. The memory can further include instructions executable by the processor to: map, using the character convolutional layer of the embedding model, each amino acid token to a convolutional latent vector of the plurality of convolutional latent vectors, the amino acid token being one-hot encoded and the convolutional latent vector being a continuous representation vector.

Training the embedding model can be achieved in an unsupervised manner using ground truth data including T cell receptor sequences. The memory can further include instructions executable by the processor to: jointly optimize a set of forward layer weights of the forward pass sub-layer and a set of backward layer weights of the backward pass sub-layer, the set of forward layer weights and the set of backward layer weights being distinct from one another.

In a further aspect, a method of generating context-aware TCR embeddings from a sequence of amino acid tokens includes: applying a sequence of amino acid tokens as input to a character convolutional layer of an embedding model resulting in a plurality of convolutional latent vectors, each convolutional latent vector of the plurality of convolutional latent vectors being respectively associated with an amino acid token of the sequence of amino acid tokens; generating a plurality of token latent vectors from the plurality of convolutional latent vectors using a bidirectional long short-term memory (LSTM) stack of the embedding model, the bidirectional LSTM stack having a plurality of bidirectional LSTM layers that collectively model a joint probability of the sequence of amino acid tokens, each token latent vector of the plurality of token latent vectors representing an amino acid token of the sequence of amino acid tokens and encoding contextual relationships between amino acid tokens of the sequence of amino acid tokens; and combining the plurality of token latent vectors into a sequence representation vector for the sequence of amino acid tokens.

The method can further include: predicting, at a softmax layer of the embedding model and based on an output of a forward pass sub-layer of a final bidirectional LSTM layer of the plurality of bidirectional LSTM layers, a next right amino acid token given one or more previous left tokens of the sequence of amino acid tokens; and predicting, at the softmax layer of the embedding model and based on an output of a backward pass sub-layer of a final bidirectional LSTM layer of the plurality of bidirectional LSTM layers, a next left amino acid token given one or more previous right tokens of the sequence of amino acid tokens. The method can further include: applying the sequence representation vector for the sequence of amino acid tokens as input to a downstream task element. The embedding model may have been trained using ground truth data including T cell receptor sequences.

The method can further include: jointly optimizing a set of forward layer weights of the forward pass sub-layer and a set of backward layer weights of the backward pass sub-layer, the set of forward layer weights and the set of backward layer weights being distinct from one another.

In a further aspect, the method can further include: training the embedding model in an unsupervised manner using ground truth data including T cell receptor sequences.

In a further aspect, a non-transitory computer readable media includes instructions encoded thereon that are executable by a processor to: apply a sequence of amino acid tokens as input to a character convolutional layer of an embedding model resulting in a plurality of convolutional latent vectors, each convolutional latent vector of the plurality of convolutional latent vectors being respectively associated with an amino acid token of the sequence of amino acid tokens; generate a plurality of token latent vectors from the plurality of convolutional latent vectors using a bidirectional long short-term memory (LSTM) stack of the embedding model, the bidirectional LSTM stack having a plurality of bidirectional LSTM layers that collectively model a joint probability of the sequence of amino acid tokens, each token latent vector of the plurality of token latent vectors representing an amino acid token of the sequence of amino acid tokens and encoding contextual relationships between amino acid tokens of the sequence of amino acid tokens; and combine the plurality of token latent vectors into a sequence representation vector for the sequence of amino acid tokens. In some examples, the embedding model has been trained using ground truth data including T cell receptor sequences.

The non-transitory computer readable media can further include instructions executable by a processor to: apply the sequence representation vector for the sequence of amino acid tokens as input to a downstream task element.

The bidirectional LSTM stack can include a forward pass sub-layer and a backward pass sub-layer for each respective bidirectional LSTM layer of the plurality of bidirectional LSTM layers. The forward pass sub-layer can have a set of forward layer weights and the backward pass sub-layer can have a set of backward layer weights that are jointly optimized during a training process of the embedding model, the set of forward layer weights and the set of backward layer weights being distinct from one another.

Each forward pass sub-layer can model a forward probability of a next right amino acid token of the sequence of amino acid tokens given one or more previous left tokens of the sequence of amino acid tokens. As such, the non-transitory computer readable media can further include instructions executable by a processor to: predict, at a softmax layer of the embedding model and based on an output of a forward pass sub-layer of a final bidirectional LSTM layer of the plurality of bidirectional LSTM layers, a next right amino acid token given one or more previous left tokens of the sequence of amino acid tokens.

Similarly, each backward pass sub-layer can model a backward probability of a next left amino acid token of the sequence of amino acid tokens given one or more previous right tokens of the sequence of amino acid tokens. As such, the non-transitory computer readable media can further include instructions executable by a processor to: predict, at a softmax layer of the embedding model and based on an output of a backward pass sub-layer of a final bidirectional LSTM layer of the plurality of bidirectional LSTM layers, a next left amino acid token given one or more previous right tokens of the sequence of amino acid tokens.

### BRIEF DESCRIPTION OF THE DRAWINGS

The present patent or application file contains at least one drawing executed in color. Copies of this patent or patent application publication with color drawing(s) will be provided by the Office upon request and payment of the necessary fee.

FIGS. 1A and 1B are a pair of simplified diagrams showing a system that implements a bi-directional amino acid embedding model;

FIG. 1C is a simplified diagram showing application of the embedding model of FIGS. 1A and 1B applied to a binding affinity prediction model;

FIG. 1D is a simplified diagram showing application of the embedding model of FIGS. 1A and 1B applied to a TCR clustering algorithm;

FIGS. 2A-2F are a series of graphical representations showing comparison of the embedding model of FIGS. 1A and 1B with state-of-the-art embedding methods for TCR-epitope binding affinity prediction task as in FIG. 1C;

FIGS. 3A-3F are a series of graphical representations showing tSNE visualization for top five frequent epitopes;

FIGS. 4A-4F are a series of graphical representations showing comparison of the amino acid embedding methods including the embedding model of FIGS. 1A and 1B for epitope-specific TCR clustering as in FIG. 1D, where FIGS. 4A-4C show NMO scores and FIGS. 4D-4F show purity scores;

FIGS. 5A and 5B are a pair of graphical representations showing TCR clustering performance for the top 34 abundant epitopes representing 70.55% of TCRs in collected databases, where hierarchical clustering is employed on embeddings computed by each method;

FIG. 6 is a simplified diagram showing an exemplary computing device for implementation of the embedding model of FIGS. 1A-1D; and

FIGS. 7A and 7B are a pair of process flow diagrams showing a process for generating context-aware TCR embeddings from a sequence of amino acid tokens that can be implemented by the computing device of FIG. 6 in accordance with the embedding model of FIGS. 1A-1D.

Corresponding reference characters indicate corresponding elements among the view of the drawings. The headings used in the figures do not limit the scope of the claims.

### DETAILED DESCRIPTION

Accurate prediction of binding interaction between T cell receptors (TCRs) and host cells is fundamental to understanding the regulation of the adaptive immune system as well as to developing data-driven approaches for personalized immunotherapy. While several machine learning models have been developed for this prediction task, the question of how to specifically embed TCR sequences into numeric representations remains largely unexplored compared to protein sequences in general. Here, the present disclosure investigates whether the embedding models designed for protein sequences, and the most widely used BLOSUM-based embedding techniques are suitable for TCR analysis. Additionally, the present disclosure presents context-aware amino acid embedding models (catELMo) designed explicitly for TCR analysis and trained on 4M unlabeled TCR sequences with no supervision. Effectiveness of catELMo in both supervised and unsupervised scenarios is evaluated by stacking the simplest models on top of the learned embeddings. For the supervised task, the binding affinity prediction problem of TCR and epitope sequences are selected and demonstrate notably significant performance gains (up by at least 14% AUC) compared to existing embedding models as well as the state-of-the-art methods. Additionally, the present disclosure also shows that the learned embeddings reduce more than 93% annotation cost while achieving comparable results to the state-of-the-art methods. In TCR clustering task (unsupervised), catELMo identifies TCR clusters that are more homogeneous and complete about their binding epitopes. Altogether, catELMo trained without any explicit supervision interprets TCR sequences better and negates the need for complex deep neural network architectures in downstream tasks.

### 1. Introduction

T cell receptors (TCRs) play critical roles in adaptive immune systems as they enable T cells to distinguish abnormal cells from healthy cells. TCRs carry this important function by binding to antigens presented by major histocompatibility complex (MHC) and recognizing whether the antigens are self or foreign. It is widely accepted that the third complementarity-determining region (CDR3) of the TCRβ chain is the most important in determining its binding specificity to epitope—a part of an antigen. The advent of publicly available databases of TCR-epitope cognate pairs opened the door to computational methods to predict the binding affinity of a given pair of TCR and epitope sequences. Computational prediction of binding affinity is important as it can drastically reduce the cost and the time needed to narrow down a set of candidate TCR targets, thereby accelerating the development of personalized immunotherapy leading to vaccine development and cancer treatment. Computational prediction is challenging primarily due to: 1) many-to-many binding characteristics; and 2) the limited amount of currently available data.

Despite the challenges, many deep neural networks have been leveraged to predict binding affinity between TCRs and epitopes. While each model has its own strengths and weaknesses, they all suffer from poor generalizability when applied to unseen epitopes, not present in the training data. In order to alleviate this, the present disclosure focuses mainly on embedding, as embedding an amino acid sequence into a numeric representation is the very first step needed to train and run a deep neural network. Furthermore, a ‘good’ embedding has been shown to boost downstream performance even with a few numbers of downstream samples.

BLOSUM matrices are widely used for representing amino acids into biological-related numeric vectors in TCR analysis. However, BLOSUM matrices are static embedding methods as they always map an amino acid to the same vector regardless of its context. For example, in static word embedding, the word “mouse” in phrases “a mouse in desperate search of cheese” and “to click, press and release the left mouse button” will be embedded as the same numeric representation even though it is used in different contexts. Similarly, the amino acid residue G appearing five times in a TCRβ CDR3 sequence CASGGTGGANTGQLYF (SEQ ID NO: 1) may play different roles in binding to antigens as each occurrence has a different position and neighboring residues. The loss of such contextual information from static embedding may inevitably compromise model performances.

Recent successes of large language models have been prompting new research applying text embedding techniques to amino acid embedding. Large language models are generally trained on a large text corpus in a self-supervised manner where no labels are required. A large number of (unlabeled) protein sequences has been available via high quality and manually curated databases such as UniProt. With the latest development of targeted sequencing assays of TCR repertoire, a large number (unlabeled) of TCR sequences has also been accessible to the public via online databases such as ImmunoSEQ. These databases have allowed researchers to develop large-scale amino acid embedding models that can be used for various downstream tasks. Asgari et al. first utilized Word2vec model with 3-mers of amino acids to learn embeddings of protein sequences. By considering a 3-mer amino acids as a word and a protein sequence as a sentence, they learn amino acid representations by predicting the context of a given target 3-mer in a large corpus of surrounding ones. Yang et al. applied Doc2vec models to protein sequences with different sizes of k-mers in a similar manner to Asgari et al. and showed better performance over sparse one-hot encoding. One-hot encoding produces static embeddings, like BLOSUM, which leads to the loss of positional and contextual information.

Later, SeqVec and ProtTrans experimented with dynamic protein sequence embeddings via multiple context-aware language models, showing advantages across multiple tasks. Note that the aforementioned amino acid embedding models were designed for protein sequence analysis. Although these models may have learned general representations of protein sequences, it does not necessarily signify their generalization performance on TCR-related downstream tasks.

Here, strategies are explored to develop amino acid embedding models, emphasizing the importance of using ‘good’ amino acid embeddings for a significant performance gain in TCR-related downstream tasks. It includes neural network depth, architecture, types and numbers of training samples, and parameter initialization. Based on experimental observation, a system is disclosed herein, referred to as “catELMo”, whose architecture is adapted from ELMo (Embeddings from Language Models), a bi-directional context-aware language model. catELMo is trained on more than four million TCR sequences collected from ImmunoSEQ in an unsupervised manner, by contextualizing amino acid inputs and predicting the next amino acid token. Performance of catELMo is compared with state-of-the-art amino acid embedding methods on two TCR-related downstream tasks. In TCR-epitope binding affinity prediction application, catELMo significantly outperforms the state-of-the-art method by at least 14% (absolute improvement) of AUCs. catELMo is also shown to achieve an equivalent performance to the state-of-the-art method while dramatically reducing downstream training sample annotation cost (more than 93% absolute reduction). In the epitope-specific TCR clustering application, catELMo also achieves comparable to or better cluster results than state-of-the-art methods.

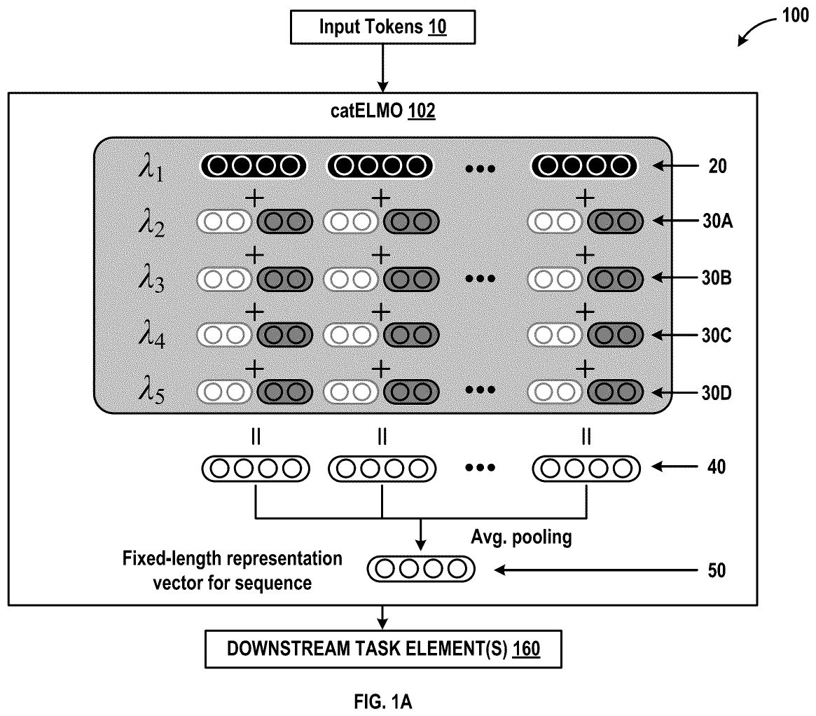

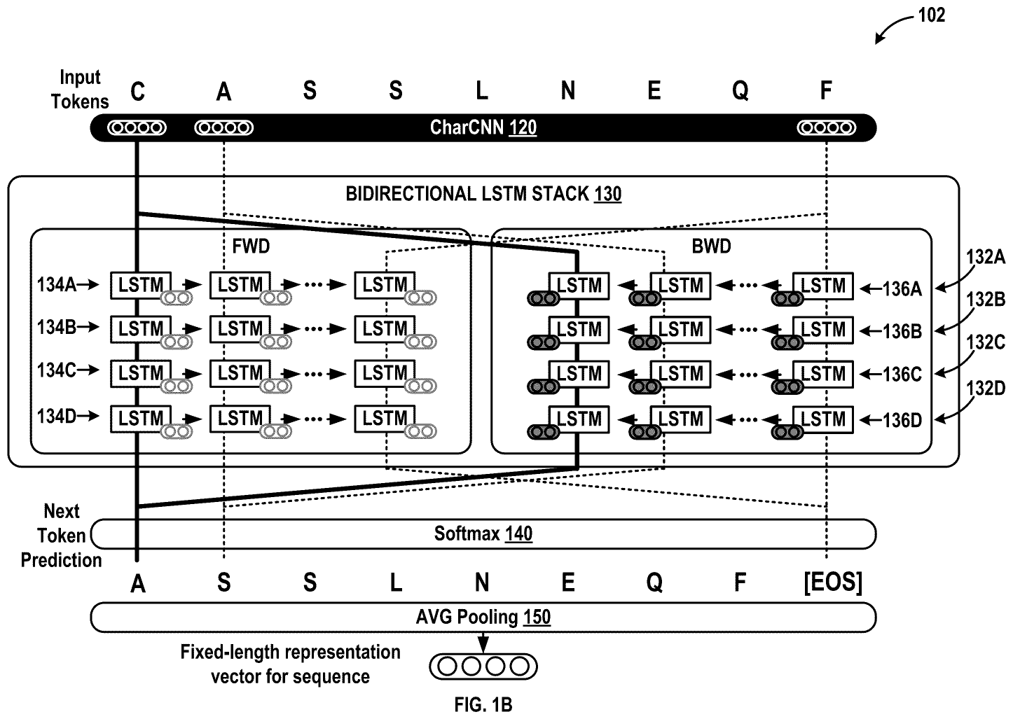

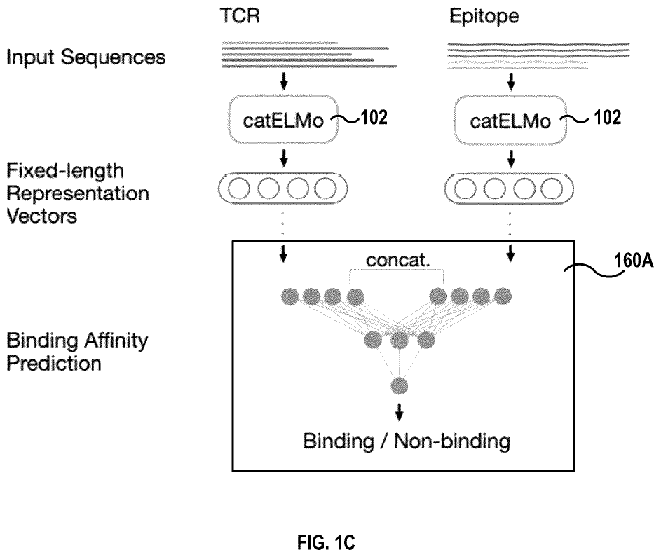

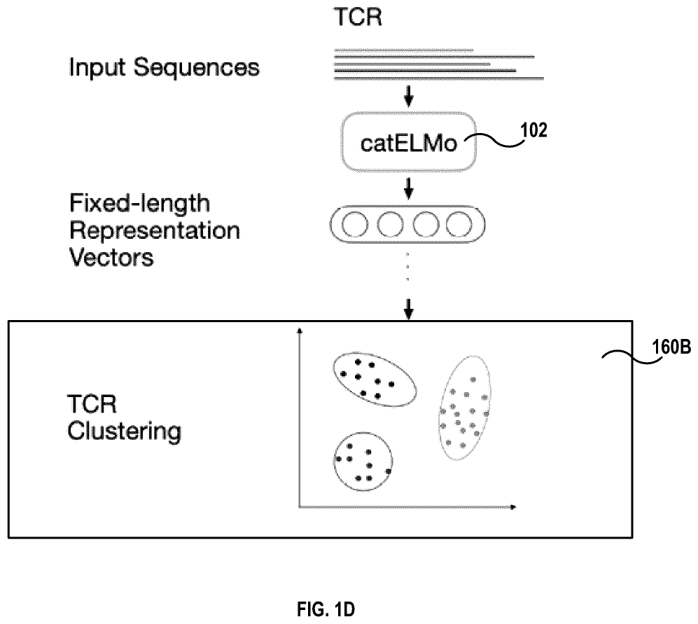

Sections 2 and 3 herein outline experimental results from benchmarking tests that compare performance of catELMo to that of other state-of-the-art methods. Section 4 outlines methods for training including data used (section 4,1) as well as an explanation of how other methods work (Section 4,2). Section 4.3 in particular outlines the present system (catELMo) with reference to FIGS. 1A-1D. Section 4.4 outlines example downstream tasks and includes information about how catELMo performs on the downstream tasks. Section 5 outlines particulars of catELMo as a computer-implemented system, including a computer-implemented method that correlates with the discussion in section 4.3.

### 2. Results

catELMo is a bi-directional amino acid embedding model that learns contextualized amino acid representations (FIGS. 1A and 1B), treating an amino acid as a word and a sequence as a sentence. It learns patterns of amino acid sequences with its self-supervision signal, by predicting each the next amino acid token given its previous tokens. It has been trained on 4,173,895 TCRβ CDR3 sequences (52 million of amino acid tokens) from ImmunoSEQ (Table 1). catELMo yields a real-valued representation vector for a sequence of amino acids, which can be used as input features of various downstream tasks. catELMo is evaluated on two different TCR-related downstream tasks, and compared its performance with existing amino acid embedding methods, namely BLOSUM62, Yang et al., ProtBert, SeqVec, and TCRBert. Various components of catELMo are also investigated in order to account for its high performance, including the neural network architecture, layer depth and size, types of training data, and the size of downstream training data.

TABLE 1
Data Summary. The number of unique epitopes, TCRs, and TCR-epitope
pairs used for catELMo and downstream tasks analysis.

| | | Unique | Unique | Unique TCR- | Amino Acid |
|---|---|---|---|---|---|
| Usage | Source | Epitopes | TCRs | epitope Pairs | Tokens |
| catELMo | ImmuneSEQ | x | 4,173,895 | x | 52,546,029 |
| Training | | | | | |
| Binding | VDJdb* | 187 | 3,915 | 4,047 | x |
| Affinity | McPAS | 301 | 9,822 | 10,156 | x |
| Prediction | IEDB | 1189 | 136,492 | 145,678 | x |
| | Total (after | 982 | 140,675 | 150,008 | x |
| | removing | | | | |
| | duplicates) | | | | |
| Epitope- | McPAS Human, | 8 | 5,607 | 5,607 | x |
| specific | Mice | | | | |
| TCR | McPAS Human | 8 | 5,528 | 5,528 | x |
| Clustering | McPAS Mice | 8 | 1,322 | 1,322 | x |

This section briefly summarizes the two downstream tasks here and refer further details to Section 4.4. The first downstream task is TCR-epitope binding affinity prediction (FIG. 1C). All embedding models compared were to embed input sequences into the identical prediction model. Each prediction model was trained on 300,016 TCR-epitope binding and non-binding pairs (1:1 ratio), embedded by each embedding model. For the binding prediction task (downstream from catELMo) a neural network with three linear layers for the prediction model, which takes a pair of TCR and epitope as input and returns a binding affinity (0-1) of the pair. The prediction performance was evaluated on testing sets each defined by two types of splitting methods, called TCR and epitope splits. The testing set of TCR split has no TCRs overlapped with training and validation sets, allowing us to measure out-of-sample TCR performance. Similarly, the testing set of epitope split has no epitopes overlapped with training and validation sets, allowing us to measure out-of-sample epitope performance. For a fair comparison, a consistent embedding method was applied to both TCR and epitope sequences within a single prediction model. The second task is epitope-specific TCR clustering that aims at grouping TCRs that bind to the same epitope (FIG. 1D), tested with TCR sequences of human and mouse species sampled from McPAS database. Hierarchical clustering is applied, and normalized mutual information (NMI) is reported to quantify the effectiveness of the clustering partition of TCR sequences.

2.1. catELMo Outperforms the Existing Embedding Methods at Discriminating Binding and Non-Binding TCR-Epitope Pairs

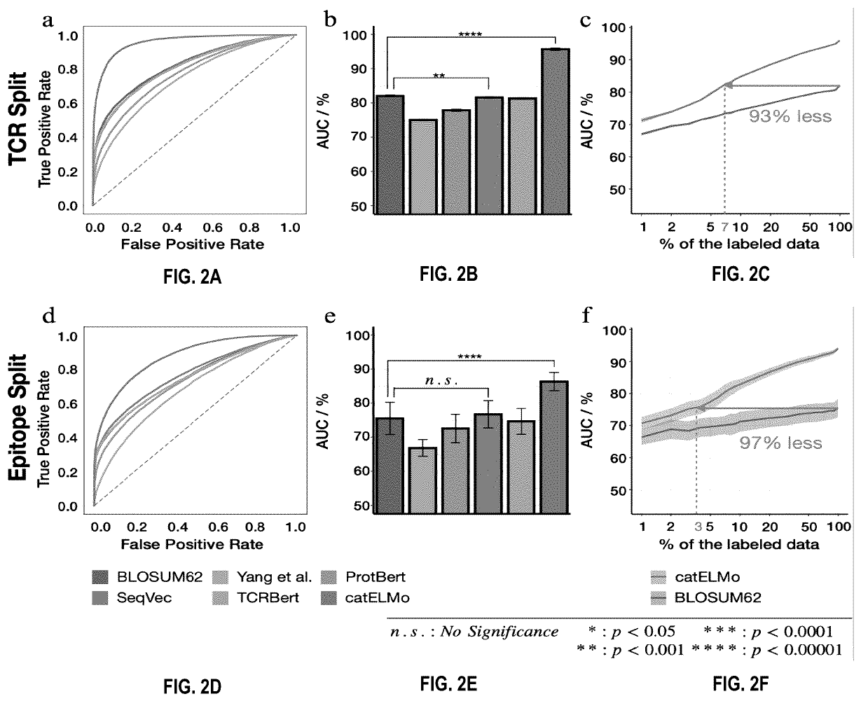

Downstream performance of TCR-epitope binding affinity prediction models trained using catELMo embeddings is investigated. In order to compare performance across different embedding methods, the identical downstream model architecture is used for each method. The competing embedding methods compared are BLOSUM62, Yang et al., ProBert, SeqVec and TCRBert. It was observed that the prediction model using catELMo embeddings significantly outperformed those using existing amino acid embedding methods in both TCR (FIGS. 2A and 2B) and epitope (FIGS. 2D and 2E) split. In TCR split, where no TCRs in the testing set exist in the training and validation set, catELMo's prediction performance was significantly greater than the second best method (p-value <6.28×10⁻²³, Table 2). It achieved AUC 96.04% which was 14 points higher than that of the second-highest performing method, while the rest of the methods performed worse than or similar to BLOSUM62. In epitope split, where no epitopes in the testing set exist in the training and validation set, the prediction model using catELMo also outperformed others with even larger performance gaps. catELMo significantly boosted 17% points of AUCs than the second-highest performing method (p-value <1.18×10⁻⁷, Table 3). Similar performance gains from catELMo were also observed in other metrics such as Precision, Recall, and F1 scores.

TABLE 2
TCR-epitope binding affinity prediction performance of
TCR split. Average and standard deviation of 10 trials
are reported. P-values are from two-sample t-tests between
catELMo and the second best method (italicised).

| | AUC (%) | Precision (%) | Recall (%) | F1 (%) |
|---|---|---|---|---|
| BLOSUM62 | 82.03 ± 0.25 | 67.16 ± 1.01 | 82.04 ± 1.01 | 70.57 ± 0.73 |
| Yang et al. | 75.03 ± 0.20 | 62.54 ± 0.78 | 79.71 ± 1.45 | 65.22 ± 0.69 |
| ProtBert | 77.86 ± 0.29 | 70.01 ± 1.47 | 69.90 ± 2.65 | 69.85 ± 0.41 |
| SeqVec | 81.61 ± 0.21 | 69.30 ± 1.33 | 79.02 ± 2.02 | 71.75 ± 0.66 |
| TCRBert | 80.79 ± 0.17 | 74.19 ± 1.17 | 70.48 ± 1.60 | 72.89 ± 0.23 |
| catELMo | 96.04 ± 0.12 | 86.88 ± 0.92 | 91.83 ± 0.98 | 88.94 ± 0.21 |
| (present | | | | |
| system) | | | | |
| p-value | 6.28 × 10⁻¹² | 1.94 × 10⁻¹⁵ | 1.82 × 10⁻¹⁴ | 1.29 × 10⁻²⁹ |

TABLE 3
TCR-epitope binding affinity prediction performance of
epitope split. Average and standard deviation of 10 trials
are reported. P-values are from two-sample t-tests between
catELMo and the second best method (italicised).

| | AUC (%) | Precision (%) | Recall (%) | F1 (%) |
|---|---|---|---|---|
| BLOSUM62 | 75.54 ± 4.74 | 64.87 ± 2.17 | 73.12 ± 5.72 | 66.65 ± 2.94 |
| Yang et al. | 66.83 ± 2.46 | 58.68 ± 1.19 | 73.00 ± 3.65 | 60.16 ± 1.52 |
| ProtBert | 72.55 ± 4.18 | 67.66 ± 3.12 | 62.21 ± 6.93 | 66.04 ± 3.23 |
| SeqVec | 76.71 ± 4.02 | 66.35 ± 2.31 | 73.49 ± 6.26 | 67.92 ± 2.74 |
| TCRBert | 74.43 ± 3.98 | 73.34 ± 2.07 | 57.02 ± 7.70 | 67.64 ± 3.45 |
| catELMo | 94.10 ± 0.90 | 84.64 ± 1.39 | 88.85 ± 2.04 | 86.33 ± 1.04 |
| (present | | | | |
| system) | | | | |
| p-value | 1.18 × 10⁻⁷ | 1.89 × 10⁻¹⁰ | 1.47 × 10⁻⁵ | 2.71 × 10⁻¹⁰ |

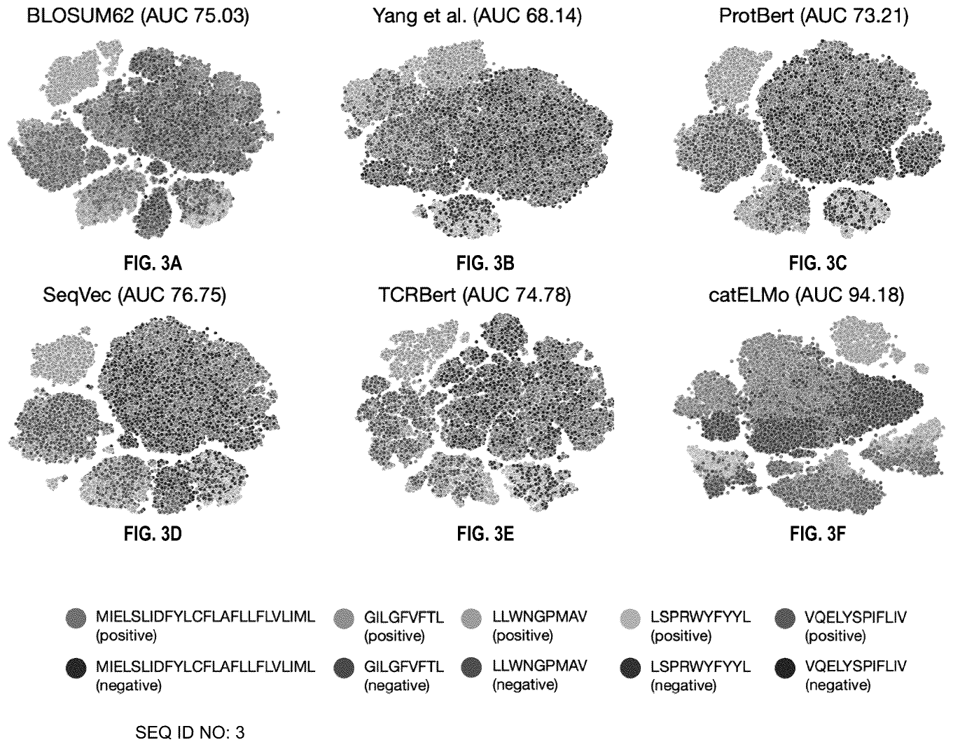

It was also visually observed that catELMo aided the model to better discriminate binding and non-binding TCRs for the five most frequent epitopes (MIELSLIDFYLCFLAFLLFLVLIML (SEQ ID NO: 2), GILGFVTFL (SEQ ID NO: 3), LLWNGPMAV (SEQ ID NO: 4), LSPRWYFYYL (SEQ ID NO: 5), VQELYSPIFLIV (SEQ ID NO: 6)) that appeared in the collected TCR-epitope pairs (FIGS. 3A-3F). These five epitopes account for a substantial portion of the dataset, comprising 14.73% (44,292 pairs) of the total TCR-epitope pairs collected. For visualization, t-SNE is performed on the top fifty principal components of the last latent vectors of each prediction model. Each point represents a pair of TCR-epitope, colored by epitope (lighter shade for positive binding and darker shade for negative binding). Different degrees of overlapping between positive pairs and negative ones in regard to the same epitope can be seen in the t-SNE plots. For example, most of the binding and non-binding data points from SeqVec embeddings are barely separated within each epitope group. On the other hand, the t-SNE plot of catELMo exhibits noticeable contrast between binding and non-binding pairs, indicating that catELMo aids the prediction model to distinguish labels. catELMo was also observed to outperform the other embedding methods in discriminating binding and non-binding TCRs for almost all individual epitopes. The prediction model using catELMo embeddings achieved the highest AUCs in 39 out of 40 epitopes, and the second highest AUC on an epitope (GTSGSPIVNR) (SEQ ID NO: 7) with only 1.09% lower than the highest score. Additionally, catELMo was observed to consistently outperform other embedding methods in predicting the binding affinity between TCRs and epitopes from a diverse range of pathogens (Table 4).

TABLE 4
AUC Scores for top 10 frequent epitope types (pathogens)
in the testing set of epitope split.

| | # of | BLOS- | Yang | | | | |
|---|---|---|---|---|---|---|---|
| Pathogens | TCRs | UM62 | et al. | ProtBert | SeqVec | TCRBert | catELMO |
| SARS-CoV-2 | 38606 | 70.32 | 65.25 | 68.16 | 71.25 | 69.85 | 95.1 |
| Influenza | 10802 | 90.66 | 76.03 | 88.77 | 91.36 | 89.86 | 95.4 |
| Yellow Fever | 4716 | 79.4 | 71.07 | 77.92 | 80.42 | 81.47 | 90.83 |
| Virus | | | | | | | |
| Human | 4646 | 73.78 | 66.32 | 70.01 | 74.58 | 72.15 | 92.36 |
| Coronavirus | | | | | | | |
| (strain SARS) | | | | | | | |
| Melanoma | 2090 | 91.05 | 78.76 | 78.43 | 89.34 | 86.54 | 94.89 |
| SARS | 2058 | 78.82 | 72.57 | 76.96 | 79.73 | 78.04 | 94.51 |
| coronavirus | | | | | | | |
| Tor2 | | | | | | | |
| Cytomegalovirus | 1104 | 76.49 | 68.87 | 72.46 | 73.58 | 70.76 | 86.19 |
| (CMV) | | | | | | | |
| Hepatitis B virus | 954 | 73.5 | 61.3 | 60.48 | 72.46 | 74.8 | 89.33 |
| (HBV) | | | | | | | |
| Neoantigen | 850 | 80 | 69.01 | 76.26 | 80.94 | 79.79 | 89.8 |
| HTLV-1 | 370 | 65.43 | 55.93 | 59.65 | 71.03 | 60.34 | 74.05 |

2.2 catELMo Reduces a Significant Amount of Annotation Cost for Achieving Comparable Prediction Power

Language models trained on large corpus are known to improve downstream task performance with a smaller number of downstream training data. Similarly in TCR-epitope binding, it is shown that catELMo trained entirely on unlabeled TCR sequences facilitates its downstream prediction model to achieve the same performance with a significantly smaller amount of TCR-epitope training pairs (i.e., epitope-labeled TCR sequence). A binding affinity prediction model was trained for each k % of downstream data (i.e., catELMo embeddings of TCR-epitope pairs) where k=1,2, . . . ,10,20,30, . . . ,100. The widely used BLOSUM62 embedding matrix was used as a comparison baseline under the same ks as it performs better than or is comparable to the other embedding methods.

A positive log-linear relationship between the number of (downstream) training data and AUCs was observed for both TCR and epitope split (FIGS. 2C and 2F). The steeper slope in catELMo suggests that prediction models utilizing catELMo embeddings exhibit higher performance gain per number of training pairs compared to the BLOSUM62-based models. In TCR split, it was observed that catELMo's binding affinity prediction models with just 7% of the training data significantly outperform ones that use a full size of BLOSUM62 embeddings (p-value=0.0032, FIG. 2C). catELMo with just 3%, 4%, and 6% of the downstream training data achieved similar performances to when using a full size of Yang et al., ProtBert, and SeqVec embeddings, respectively. Similarly, in epitope split, it was shown that catELMo's prediction models with just 3% of training data achieved equivalent performance as ones built on a full size of BLOSUM62 embeddings (p-value=0.8531, FIG. 2F). Compared to the other embedding methods, catELMo with just 1%, 2%, and 5% of the downstream training data achieved similar or better performance than when using a full size of Yang et al., ProtBert, and SeqVec embeddings, separately. Similar performance gains from catELMo were also observed in other metrics such as Precision, Recall, and F1 scores. Achieving accurate prediction with a small amount of training data is important for TCR analysis as obtaining the binding affinity of TCR-epitope pairs is costly.

2.3 catELMo Allows Clustering of TCR Sequences with High Performance

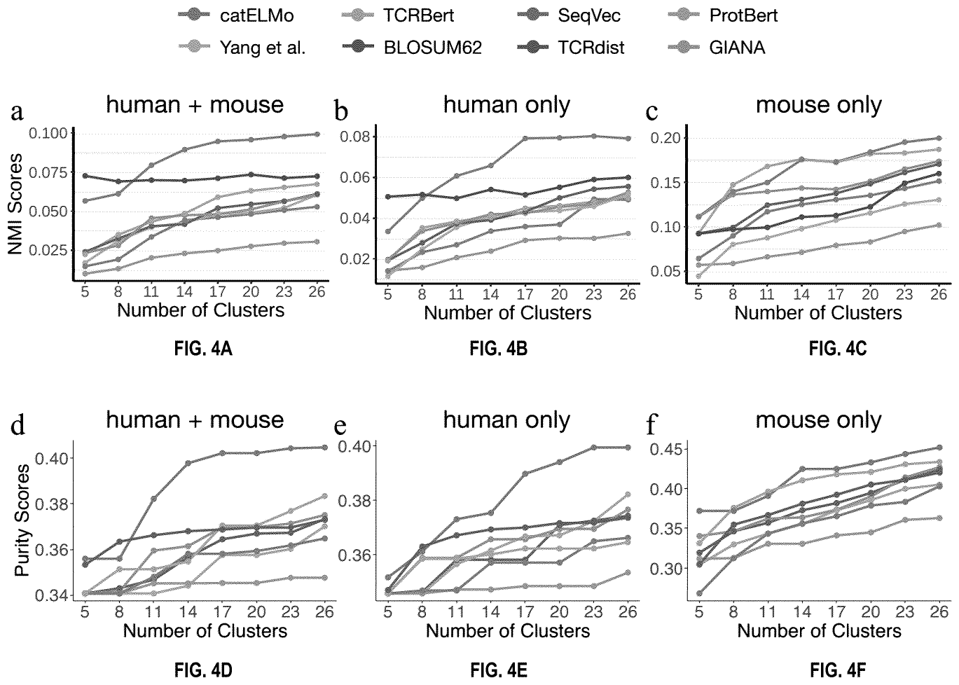

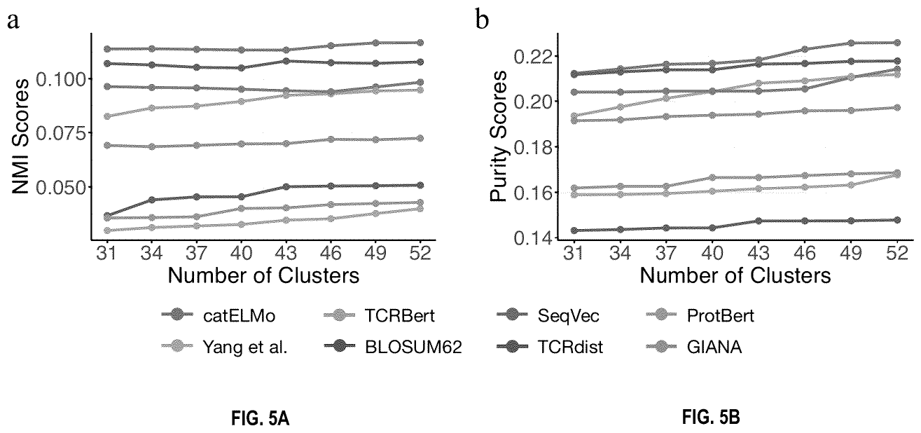

Clustering TCRs of similar binding profiles is important in TCR repertoire analysis as it facilitates discoveries of TCR clonotypes that are condition-specific. In order to demonstrate that catELMo embeddings can be used for other TCR-related downstream tasks, hierarchical clustering was performed using each method's embedding (catELMo, BLOSUM62, Yang et al., ProBert, SeqVec and TCRBert) and the identified clusters were evaluated against the ground-truth TCR groups labeled by their binding epitopes. The results were additionally compared with state-of-the-art TCR clustering methods, TCRdist and GIANA, both of which were developed from BLOSUM62 matrix (see Section 4.4.2). Normalized mutual information (NMI) and cluster purity are used to measure the clustering quality. Significant disparities in TCR binding frequencies exist across different epitopes. To construct more balanced clusters, TCR sequences were targeted that were bound to the top eight frequent epitopes identified in the McPAS database. FIGS. 4A and 4D demonstrate NMI clustering comparison results of all TCR sequences bound to the top eight epitopes, covering both human and mouse species. It was found that the cluster model built on catELMo embeddings maintains either the best or second best NMI scores compared with ones that are computed on other embeddings. To investigate whether this observation remains true on individual species, the same clustering analysis is conducted on human and mouse species, separately. FIGS. 4B and 4C showcase NMI comparison for the top eight epitopes in human (FIGS. 4B and 4E) and mouse (FIGS. 4C and 4F) species, observing a similar pattern that clustering results with catELMo achieve the highest or second-highest NMI and purity scores. Similar performance gains were observed in FIGS. 5A and 5B. Altogether, catELMo embedding can assist TCR clustering with no supervision while achieving similar or better performance than other state-of-the-art methods in both human and mouse species.

### 2.4 ELMo-Based Architecture is Preferable to BERT-Based Architecture in TCR Embedding Models

It was observed that catELMo using ELMo-based architecture outperformed the model using embeddings of TCRBert which uses BERT (Table 5). The performance differences were approximately 15% AUCs in TCR split (p-value<3.86×10⁻³⁰) and 19% AUCs in epitope split (p-value<3.29×10⁻⁸). Because TCRBert was trained on a smaller amount of TCR sequences (around 0.5 million sequences) than catELMo, catELMo is further compared with various sizes of BERT-like models trained on the same dataset as catELMo: BERT-Tiny-TCR, BERT-Base-TCR, and BERT-Large-TCR having a stack of 2, 12, and 30 Transformer layers respectively (see Section 4.6.2 for more details). Note that BERT-Base-TCR uses the same number of Transformer layers as TCRBert. Additionally, different versions of catELMo are compared by varying the number of BILSTM layers (2, 4-default, and 8, see Section 4.6.1 for more details). As summarized in Table 5, TCR-epitope binding affinity prediction models trained on catELMo embeddings (AUC 96.04% and 94.70% on TCR and epitope split) consistently outperformed models trained on these Transformer-based embeddings (AUC 81.23-81.91% and 74.20-74.94% on TCR and epitope split). The performance gaps between catELMo and Transformer-based models (14% AUCs in TCR split and 19% AUCs in epitope split) were statistically significant (p-values <6.72×10⁻²⁶ and <1.55×10⁻⁷ for TCR and epitope split respectively). It is observed that TCR-epitope binding affinity prediction models trained on catELMo-based embeddings consistently outperformed the ones using Transformer-based embeddings (Table 5, 6). Even the worst-performed BILSTM-based embedding model achieved higher AUCs than the best-performed Transformer-based embeddings at discriminating binding and non-binding TCR-epitope pairs in both TCR (p-value<2.84×10⁻²⁸) and epitope split (p-value<5.86×10⁻⁶).

TABLE 5
AUCs of TCR-epitope binding affinity prediction models built on BERT-based
embedding models. Average and standard deviation of 10 trials are reported.

| | Nums of | | | |
|---|---|---|---|---|
| | Transformer | Embedding | TCR Split | Epitope Split |
| | Layers | Size | (%) | (%) |
| BERT-Tiny-TCR | 2 | 768 | 81.23 ± 0.18 | 74.20 ± 4.01 |
| BERT-Base-TCR | 12 | 768 | 81.91 ± 0.21 | 74.94 ± 4.49 |
| BERT-Large-TCR | 30 | 1,024 | 81.29 ± 0.17 | 74.65 ± 3.79 |
| TCRBert | 12 | 768 | 80.79 ± 0.17 | 74.43 ± 3.98 |

TABLE 6
AUCs of TCR-epitope binding affinity prediction models trained
on different sizes of catELMo embeddings. Average and standard
deviation of 10 trials are reported.

| | Nums of | | | |
|---|---|---|---|---|
| | BILSTM | Embedding | TCR Split | Epitope Split |
| | Layers | Size | (%) | (%) |
| catELMo- | 2 | 1,024 | 95.67 ± 0.32 | 86.32 ± 2.68 |
| Shallow | | | | |
| catELMo | 4 | 1,024 | 96.04 ± 0.12 | 94.10 ± 0.90 |
| catELMo-Deep | 8 | 1,024 | 93.94 ± 0.19 | 91.57 ± 1.59 |

2.5 within-Domain Transfer Learning is Preferable to Cross-Domain Transfer Learning in TCR Analysis

catELMo, trained on TCR sequences, significantly outperformed amino acid embedding methods trained on generic protein sequences. catELMo-Shallow and SeqVec shared the same architecture including character-level convolutional layers and a stack of two bi-directional LSTM layers but were trained on different types of training data. catELMo-Shallow was trained on TCR sequences (about 4 million) while SeqVec was trained on generic protein sequences (about 33 million). Although catELMo-Shallow was trained on a relatively smaller amount of sequences compared to SeqVec, the binding affinity prediction model built on catELMo-Shallow embeddings (AUC 95.67% in TCR split and 86.32% in epitope split) significantly outperformed the one built on SeqVec embeddings (AUC 81.61% in TCR split and 76.71% in epitope split) by 14.06% and 9.61% on TCR and epitope split respectively. This suggests that knowledge transfer within the same domain is preferred whenever possible in TCR analysis.

### 3. Discussion

catELMo is an effective embedding model that brings substantial performance improvement in TCR-related downstream tasks. This study emphasizes the importance of choosing the right embedding models. The embedding of amino acids into numeric vectors is the very first and crucial step that enables the training of a deep neural network. It has been previously demonstrated that a well-designed embedding can lead to significantly improved results on downstream analysis. The reported performance of catELMo embedding on TCR-epitope binding affinity prediction and TCR clustering tasks indicates that catELMo is able to learn patterns of amino acid sequences more effectively than state-of-the-art embedding methods. While all other methods compared (except BLOSUM62) leverage a large number of unlabeled amino acid sequences, only the prediction model using catELMo significantly outperforms widely used BLOSUM62 and other models such as netTCR and ATM-TCR trained on paired (TCR-epitope) samples only (Table 7). This work suggests the need for developing sophisticated strategies to train amino acid embedding models that can enhance the performance of TCR-related downstream tasks even while requiring less amount of data and simpler prediction model structures.

TABLE 7
AUCs of TCR-epitope binding affinity prediction models
comparison with state-of-the-art prediction models.
All models are trained on the same dataset. Average
and standard deviation of 10 trials are reported.

| | TCR Split | Epitope Split |
|---|---|---|
| | (%) | (%) |
| catELMo | 96.04 ± 0.12 | 94.10 ± 0.90 |
| ATM-TCR | 80.92 ± 0.26 | 74.87 ± 0.64 |
| netTCR | 81.07 ± 0.31 | 73.70 ± 0.44 |

Two important observations made from the experiments are: 1) the type of data used for training amino acid embedding models is far more important than the amount of data; and 2) ELMo-based embedding models consistently perform much better than BERT-based embedding models. While previously developed amino acid embedding models such as SeqVec and ProtBert were respectively trained on 184-times and 1,690-times more amino acid tokens compared to the training data used for catELMo, the prediction models using SeqVec and ProtBert performed poorly compared to the model using catELMo (see Sections 2.1 and 2.3). SeqVec and ProtBert were trained based on generic protein sequences, whereas catELMo was trained on a collection of TCR sequences from pooled TCR repertoires across many samples, indicating that the use of TCR data to train embedding models is more critical than much larger amount of generic protein sequences.

In the field of natural language processing, Transformer-based models have been bolstered as the superior embedding model. However, for TCR-related downstream tasks, catELMo using biLSTM layer-based design outperforms BERT using Transformer layers (see Section 2.4). While it is difficult to pinpoint the reasons, the bi-directional architecture to predict the next token based on its previous tokens in ELMo may mimic the interaction process of TCR and epitope sequences either from left to right or from right to left. In contrast, BERT uses Transformer encoder layers that attend tokens both on the left and right to predict a masked token, refer to as masked language modeling. As the Transformer layer can be along with the next token prediction objectives, it remains as a future work to investigate Transformer with causal language models, such as GPT-3, for amino acid embedding. Additionally, the clear differences of TCR sequences compared to natural languages are 1) the compact vocabulary size (20 standard amino acids vs. over 170k English words) and 2) the length of peptides in TCRs being smaller than the number of words in sentences or paragraphs in natural languages. These differences may allow catELMo to learn sequential dependence without losing long-term memory from the left end.

Often in classification problems in life sciences, the difference in the number of available positive and negative data can be very large and TCR-epitope binding affinity prediction problem is no exception. In fact, the number of experimentally generated non-binding pairs are practically non-existent and obtaining experimental negative data is costly. This requires researchers to come up with a strategy to generate negative samples and it can be non-trivial. A common practice is to sample new TCRs from repertoires and pair them with existing epitopes, a strategy also employed here. Another approach is to randomly shuffle TCR-epitope pairs within positive binding dataset, resulting in TCRs and epitopes that are not known to bind paired together. Given the vast diversity of human TCR clonotypes, which can exceed 10¹⁵, the chance of randomly selecting a TCR that specifically recognizes a target epitope is relatively small. The prediction model consistently outperformed the other embedding methods by large margins in both TCR and epitope splits. The model using catELMo achieves 24% and 36% higher AUCs over the second best embedding method for TCR (p-value<1.04×10⁻¹⁸) and epitope (p-value<6.26×10⁻¹⁴) split, respectively. Moreover, it is observed that using catELMo embeddings, prediction models that are trained with only 2% downstream samples still statistically outperform ones that are built on a full size of BLOSUM62 embeddings in TCR split (p-value=0.0005). Similarly, with only 1% training samples, catELMo reaches comparable results as BLOSUM62 with a full size of downstream samples in epitope split (p-value=0.1438). In other words, catELMo dramatically reduces about 98% annotation cost. To mitigate potential batch effects, new negative pairs were generated using different seeds. Consistent prediction performance is observed across these variations. Experimental results confirm that the embeddings from catELMo maintain high performance regardless of the methodology used to generate negative samples.

Parameter fine-tuning in neural networks is a training scheme where initial weights of the network are set to the weights of a pre-trained network. Fine-tuning has been shown to bring performance gain to the model over using random initial weights. The possibility of performance boost of the prediction model using fine-tuned catELMo was investigated. Since SeqVec shares the same architecture with catELMo-Shallow and is trained on generic protein sequences, the weights of SeqVec were used as initial weights when fine-turning catELMo-Shallow. The performance of binding affinity prediction models was compared using the fine-tuned catELMo-Shallow and vanilla catELMo-Shallow (trained from scratch with random initial weights from a standard normal distribution). It is observed that the performance when using fine-tuned catELMo-Shallow embeddings was significantly improved by approximately 2% AUCs in TCR split (p-value<4.17×10⁻⁹) and 9 points AUCs in epitope split (p-value<5.46×10⁻⁷).

While epitope embeddings are a part of the prediction models outlined herein, their impact on overall performance appears to be less significant compared to that of TCR embeddings. To understand the contribution of epitope embeddings, additional experiments were performed. First, epitope embeddings were kept unchanged using the widely-used BLOSUM62 database while varying different embeddings methods exclusively for TCRs. The results (Table 8) closely align with previous findings (tables 2 and 3), suggesting that the choice of epitope embedding method may not strongly affect the final predictive performance.

TABLE 8
AUCs of TCR-epitope binding affinity prediction models
with BLOSUM62 to embed epitope sequences. The average
and standard deviation of 10 trials are reported.

| | Epitope | TCR Split | Epitope Split |
|---|---|---|---|
| TCR Embedding | Embedding | (%) | (%) |
| BLOSUM62 | BLOSUM62 | 82.03 ± 0.25 | 75.54 ± 4.74 |
| Yang et al. | BLOSUM62 | 74.85 ± 0.06 | 69.12 ± 0.24 |
| ProtBert | BLOSUM62 | 78.02 ± 0.07 | 72.1 ± 0.48 |
| SeqVec | BLOSUM62 | 81.67 ± 0.19 | 76.3 ± 0.22 |
| TCRBert | BLOSUM62 | 81.07 ± 0.12 | 74.41 ± 0.42 |
| catELMo | BLOSUM62 | 96.30 ± 0.15 | 94.46 ± 1.08 |

Furthermore, alternative embedding approaches for epitope sequences were investigated. Specifically, epitope embeddings were replaced with randomly initialized matrices containing trainable parameters, while catELMo was employed for TCR embeddings. This setting yielded predictive performance comparable to the scenario where both TCR and epitope embeddings were catELMo-based (Table 9).

TABLE 9
AUCs of TCR-epitope binding affinity prediction models trained on
catELMo TCR embeddings and random-initialized epitope embeddings.
The average and standard deviation of 10 trials are reported.

| | Epitope | TCR Split | Epitope Split |
|---|---|---|---|
| TCR Embedding | Embedding | (%) | (%) |
| catELMo | catELMo | 96.04 ± 0.12 | 94.10 ± 0.90 |
| catELMo | Randomization | 96.19 ± 0.09 | 94.61 ± 0.11 |

Similarly, using BLOSUM62 for TCR embeddings and catELMo for epitope embeddings resulted in performance similar to when both embeddings were based on BLOSUM62. These consistent findings support the proposition that the influence of epitope embeddings may not be as significant as that of TCR embeddings (Table 10).

TABLE 10
AUCs of TCR-epitope binding affinity prediction models
trained on catELMo and BLOSUM62 embeddings. The average
and standard deviation of 10 trials are reported.

| | Epitope | TCR Split | Epitope Split |
|---|---|---|---|
| TCR Embedding | Embedding | (%) | (%) |
| catELMo | catELMo | 96.04 ± 0.12 | 94.10 ± 0.90 |
| catELMo | BLOSUM62 | 96.30 ± 0.15 | 94.46 ± 1.08 |
| BLOSUM62 | catELMo | 82.16 ± 0.17 | 75.30 ± 4.49 |
| BLOSUM62 | BLOSUM62 | 82.03 ± 0.25 | 75.54 ± 4.74 |

It is believed that these observations may be attributed to the substantial data scale discrepancy between TCRs (more than 290k) and epitopes (less than 1k). Moreover, TCRs tend to exhibit high similarity, whereas epitopes display greater distinctiveness from one another. These features of TCRs require robust embeddings to facilitate effective separation and improve downstream performance, while epitope embeddings primarily serve as categorical encodings.

While TCRβ CDR3 is known to be the primary determinant for TCR-epitope binding specificity, other regions such as CDR1 and CDR2 on TCRβ V gene along with TCRα chain are also known to contribute to specificity in antigen recognition. However, the present disclosure focuses on modeling CDR3 of TCRβ chains because of the limited availability of sample data from other regions. Future work may explore strategies to incorporate these regions while mitigating the challenges of working with limited samples.

### 4. Methods

This section first presents data used for training the amino acid embedding models and the downstream tasks, and then reviews existing amino acid embedding methods and their usage on TCR-related tasks. This section also outlines the present system, catELMo, which is a bi-directional amino acid embedding method that computes contextual representation vectors of amino acids of a TCR (or epitope) sequence. This section describes in detail how to apply catELMo to two different TCR-related downstream tasks, and provides details on the experimental design, including the methods and parameters used in comparison and ablation studies.

### 4.1 Data

TCRs for training catELMo: 5,893,249 TCR sequences were collected from repertoires of seven projects in the ImmunoSEQ database: HIV, SARS-CoV2, Epstein Barr Virus, Human Cytomegalovirus, Influenza A, Mycobacterium Tuberculosis, and Cancer Neoantigens. CDR3 sequences of TCRβ chains were used to train the amino acid embedding models as those are the major segment interacting with epitopes and exist in large numbers. Duplicated copies and sequences containing wildcards such as ‘*’ or ‘X’ were excluded. Altogether, 4,173,895 TCR sequences (52,546,029 amino acid tokens) were obtained, of which 85% were used for training and 15% were used for testing.

TCR-epitope pairs for binding affinity prediction: TCR-epitope pairs known to bind each other were collected from three publicly available databases: IEDB, VDJdb, and McPAS. Unlike the (unlabeled) TCR dataset for catELMo training, each TCR is annotated with an epitope known to bind each other, which are referred to as a TCR-epitope pair. Only pairs with human MHC class I epitopes and CDR3 sequences of the TCRβ chain were used, and sequences containing wildcards such as ‘*’ or ‘X’ were filtered out. For VDJdb, pairs with a confidence score of 0 were excluded as it means a critical aspect of sequencing or specificity validation is missing. Duplicated copies were removed and datasets collected from the three databases were merged. For instance, 29.85% of pairs from VDJdb overlapped with IEDB, and 55.41% of pairs from McPAS overlapped with IEDB. Altogether, 150,008 unique TCR-epitope pairs known to bind to each other were obtained, having 140,675 unique TCRs and 982 unique epitopes. The same number of non-binding TCR-epitope pairs were generated as negative samples by randomly pairing each epitope of the positive pairs with a TCR sampled from the healthy TCR repertoires of ImmunoSEQ. Note that this includes no identical TCR sequences with the TCRs used for training the embedding models. Altogether, 300,016 TCR-epitope pairs were obtained, where 150,008 pairs are positive and 150,008 pairs are negative. The average length of TCRs and epitope sequences are 14.78 and 11.05, respectively Data collection and preprocessing procedures closely followed those outlined in Cai et al.

TCRs for antigen-specific TCR clustering: 9,822 unique TCR sequences of humans and mice hosts were collected from McPAS. Each TCR is annotated with an epitope known to bind, which is used as a ground-truth label for TCR clustering. TCR sequences that bind to neoantigen pathogens or multiple epitopes were excluded, and only included CDR3 sequences of TCRβ chain. Three subsets were composed for different experimental purposes. The first dataset includes both human and mice TCRs. TCRs associated with the top eight frequent epitopes were used, resulting in 5,607 unique TCRs. The second dataset includes only human TCRs, and the third dataset includes only mouse TCRs. In a similar manner, TCRs that bind to the top eight frequent epitopes were selected. As a result, 5,528 unique TCR sequences were obtained for the second dataset and 1,322 unique TCR sequences were obtained for the third dataset.

### 4.2 Amino Acid Embedding Methods

This section reviews previously-proposed amino acid embedding methods. There are two categories of the existing approaches: static and context-aware embedding methods. Static embedding method represents an amino acid as a static representation vector remaining the same regardless of its context. Context-aware embedding method, however, represents an amino acid differently in accordance with its context. Context-aware embedding is also called dynamic embedding in contrast to static embedding. The key ideas of various embedding methods are explained herein.

### 4.2.1 Static Embeddings

BLOSUM. BLOSUM is a scoring matrix where each element represents how likely an amino acid residue is to be substituted by another over evolutionary time. It has been commonly used to measure alignment scores between two protein sequences. There are various BLOSUM matrices such as BLOSUM45, BLOSUM62, and BLOSUM80 where a matrix with a higher number is used for the alignment of less divergent sequences. BLOSUM have also served as the de facto standard embedding method for various TCR analyses. For example, BLOSUM62 was used to embed TCR and epitope sequences for training deep neural network models predicting their binding affinity. BLOSUM62 was also used to embed TCR sequences for antigen-specific TCR clustering and TCR repertoire clustering. GIANA clustered TCRs based on the Euclidean distance between TCR embeddings. TCRdist used BLOSUM62 matrix to compute the dissimilarity matrix between TCR sequences for clustering.

Word2vec and Doc2vec. Word2vec and Doc2vec are a family of embedding models to learn a single linear mapping of words, which takes a one-hot word indicator vector as input and returns a real-valued word representation vector as output. There are two types of Word2vec architectures: continuous bag-of-words (CBOW) and skip-gram. CBOW predicts a word from its surrounding words in a sentence. It embeds each input word via a linear map, sums all input words' representations, and applies a softmax layer to predict an output word. Once training is completed, the linear mapping is used to obtain a representation vector of a word. On the contrary, skip-gram predicts the surrounding words given a word while it also uses a linear mapping to obtain a representation vector. Doc2vec is a model further generalized from Word2vec, which introduces a paragraph vector representing paragraph identity as an additional input. Doc2vec also has two types of architectures: distributed memory (DM) and distributed bag-of-words (DBOW). DM predicts a word from its surrounding words and the paragraph vector, while DBOW uses the paragraph vector to predict randomly sampled context words. In a similar way, linear mapping is used to obtain a continuous representation vector of a word.

Several studies adapted Word2vec and Doc2vec to embed amino acid sequences. ProtVec is the first Word2vec representation model trained on a large number of amino acid sequences. Its embeddings were used for several downstream tasks such as protein family classification, disordered protein visualization, and classification. Kimothi et al. adapted Doc2vec to embed amino acid sequences for protein sequence classification and retrieval. Yang et al. trained Doc2vec models on 524,529 protein sequences of UniProt database. They considered a k-mer amino acids as a word, and a protein sequence as a paragraph. They trained DM models to predict a word from w surrounding words and a paragraph with various sizes of k and w.

### 4.2.2 Context-Aware Embeddings

ELMo. ELMo is a deep context-aware word embedding model trained on a large corpus. It learns each token's (e.g., a word) contextual representation in forward and backward directions using a stack of two bi-directional LSTM layers. Each word of a text string is first mapped into a numerical representation vector via the character-level convolutional layers. The forward (left-to-right) pass learns a token's contextual representation depending on itself and the previous context in which it is used. The backward (left-to-right) pass learns a token's representation depending on itself and its subsequent context.

ELMo is less commonly implemented for amino acid embedding than Transformer-based deep neural networks. One example is SeqVec. It is an amino acid embedding model using ELMo's architecture. It feeds each amino acid as a training token of size 1, and learns its contextual representation both forward and backward within a protein sequence. The data was collected from UniRef50, which includes 9 billion amino acid tokens and 33 million protein sequences. SeqVec was applied to several protein-related downstream tasks such as secondary structure and long intrinsic disorder prediction, and subcellular localization.

BERT. BERT is a large language model leveraging Transformer layers to learn context-aware word embeddings jointly conditioned on both directions. BERT is learned for two objectives. One is the masked language model to learn contextual relationships between words in a sentence. It aims to predict the original value of masked words. The other is the next sentence prediction which aims to learn the dependency between consecutive sentences. It feeds a pair of sentences as input and predicts whether the first sentence in the pair is contextually followed by the second sentence.

BERT's architecture has been used in several amino acid embedding methods. They treated an amino acid residue as a word and a protein sequence as a sentence. ProtBert was trained on 216 million protein sequences (88 billion amino acid tokens) of UniRef100. It was applied for several protein sequence applications such as secondary structure prediction and sub-cellular localization. ProteinBert combined language modeling and gene ontology annotation prediction together during training. It was applied to protein secondary structure, remote homology, fluorescence and stability prediction. TCRBert was trained on 47,040 TCRβ and 4,607 TCRα sequences of PIRD dataset and evaluated on TCR-antigen binding prediction and TCR engineering tasks.

4.3 catELMo

Referring to FIGS. 1A-1D, the present disclosure outlines a system 100 including an embedding model 102 referred to herein as “catELMo”, a bi-directional amino acid embedding model designed for TCR analysis. The embedding model 102 adapts ELMo's architecture to learn context-aware representations of amino acids. It is trained on TCR sequences, which is different from the existing amino acid embedding models such as SeqVec trained on generic protein sequences. FIG. 1A shows the embedding model 102 in the context of the system 100 with various data types, and FIG. 1B shows the embedding model 102 including its constituent components. As illustrated in FIGS. 1A and 1B, the embedding model 102 includes a character CNN (CharCNN) layer 120 that receives a sequence of amino acid tokens 10 as input and converts each one-hot encoded amino acid token to a convolutional latent vector 20, which is a continuous representation vector. The embedding model 102 also includes a bidirectional LSTM stack 130 that includes four bi-directional LSTM layers 132A-132D. The bidirectional LSTM stack 130 learns contextual relationships between amino acid residues encoded within the sequence of amino acid tokens 10, where each layer of the bidirectional LSTM stack 130 produces a LSTM latent vector 30A, 30B, 30C or 30D for each amino acid token. The embedding model 102 further includes a softmax layer 140 at an output of the bidirectional LSTM stack 130 that predicts the next (or previous) amino acid token and produces a plurality of token latent vectors 40 (one for each amino acid token). Following the softmax layer 140, the embedding model 102 can include an average pooling layer 150 that combines the plurality of token latent vectors 40 into a sequence representation vector 50. The sequence representation vector 50 may be applied as input to a downstream task element 160 that performs a downstream task, such as a binding affinity task element 160A shown in FIG. 1C or a TCR clustering task element 160B shown in FIG. 1D.

Given a sequence of N amino acid tokens, (t₁, t₂, . . . , t_{N}) (e.g., sequence of amino acid tokens 10, shown in FIG. 1B as sequence CASSLNEQF (SEQ ID NO: 8)), the CharCNN 120 of the embedding model 102 maps each one-hot encoded amino acid token tₖ to a latent vector cₖ (e.g., a convolutional latent vector of a plurality of convolutional latent vectors 20) through seven convolutional layers with kernel sizes ranging from 1 to 7, and the numbers of filters of 32, 32, 64, 128, 256, 512, and 512 (or 1024), each of which is followed by a max-pooling layer, resulting in a 1,024-dimensional vector. The output of the CharCNN, (c₁, c₂, . . . , c_{N}), is then fed into a stack of four bidirectional LSTM layers (e.g., the bidirectional LSTM stack 130, which includes four bi-directional LSTM layers 132A-132D) including forward pass sub-layers 134A-134D and backward pass sub-layers 136A-136D (e.g., where each respective bidirectional LSTM layer 132A, 132B, 132C or 132D includes a forward pass sub-layer 134A, 134B, 134C or 134D and a backward pass sub-layer 136A, 136B, 136C or 136D). For the forward pass, the sequence of the CharCNN output (e.g., the plurality of convolutional latent vectors 20) is fed into a first forward LSTM layer (e.g., a first forward pass sub-layer 134A) followed by a second forward LSTM layer (e.g., a second forward pass sub-layer 134B), and so on. Each LSTM cell in every forward layer has 4,096 hidden states and returns a 512-dimensional representation vector. Each output vector of the final LSTM layer is then fed into the softmax layer 140 to predict the right next amino acid token. Residual connection is applied between the first and second layers and between the third and fourth layers to prevent gradient vanishing. Similarly, the sequence of the CharCNN output (e.g., the plurality of convolutional latent vectors 20) is fed into the backward pass sub-layers 136A-136D, in which each cell returns a 512-dimensional representation vector. Unlike the forward layer, each output vector of the backward layer followed by a softmax layer aims to predict the left next amino acid token.

Through the forward and backward passes, the embedding model 102 models the joint probability of a sequence of amino acid tokens. The forward pass aims to predict the next right amino acid token given its left previous tokens, which is P(tₖ|t₁, t₂, . . . , tₖ₋₁; θ_{c}, θ_{fw}, θₛ) for each k-th cell where θ_{c} indicates parameters of CharCNN, θ_{fw} indicates parameters of the forward layers, and θₛ indicates parameters of the softmax layer. The joint probability of all amino acid tokens for the forward pass is defined as:

$$\\ P ( {t}_{1} , {t}_{2} , … , {t}_{N} ; {θ}_{c} , {θ}_{fw} , {θ}_{s} ) = {\prod }_{k = 1}^{N} P ( {t}_{k} \vert {t}_{1} , {t}_{2} , … , {t}_{k - 1} ; {θ}_{c} , {θ}_{fw} , {θ}_{s} )$$

The backward pass aims to predict the next left amino acid token given its right previous tokens. Similarly, the joint probability of all amino acid tokens for the backward pass is defined as:

$$\\ P ( {t}_{1} , {t}_{2} , … , {t}_{N} ; {θ}_{c} , {θ}_{bw} , {θ}_{s} ) = {\prod }_{k = 1}^{N} P ( {t}_{k} \vert {t}_{k + 1} , {t}_{k + 2} , … , {t}_{N} ; {θ}_{c} , {θ}_{bw} , {θ}_{s} )$$

where θ_{bw} indicates parameters of the backward layers. During training of the embedding model 102, the combined log-likelihood of the forward and backward passes is jointly optimized, which is defined as:

$$\\ {\sum }_{k = 1}^{N} [ \begin{matrix}log P ( {t}_{k} \vert {t}_{1} , {t}_{2} , … , {t}_{k - 1} ; {θ}_{c} , {θ}_{fw} , {θ}_{s} ) + \\ log P ( {t}_{k} \vert {t}_{k + 1} , {t}_{k + 2} , … , {t}_{N} ; {θ}_{c} , {θ}_{bw} , {θ}_{s} )\end{matrix} ] .$$

Note that the forward and backward layers have their own weights (θ_{fw} and θ_{bw}). This helps to avoid information leakage that a token used to predict its right tokens in forward layers is undesirably used again to predict its own status in backward layers.

For each amino acid residue, the embedding model 102 computes five representation vectors of length 1,024: one convolutional latent vector 20 from CharCNN and four LSTM latent vectors 30A-30D from the four bi-directional LSTM layers 132A-132D. For a given TCR sequence of length L, each layer returns L vectors of length 1024. The size of an embedded TCR sequence, therefore, is [5, L, 1024]. Those vectors are averaged over and yield an amino acid representation vector (e.g., a token latent vector 40) of length 1,024. A sequence of amino acids is then represented by an element-wise average of all amino acids' representation vectors, resulting in a sequence representation vector 50 of length 1,024. For example, the embedding model 102 computes a representation for each amino acid in a TCR sequence, e.g., CASSPTSGGQETQYF (SEQ ID NO: 9), as a vector of length 1,024. The sequence is then represented by averaging over 15 amino acid representation vectors, which is a vector with a length of 1,024. The embedding model 102 is trained up to 10 epochs with a batch size of 128 on two NVIDIA RTX 2080 GPUs. The default experimental settings of ELMo are followed unless otherwise specified. In some examples, the embedding model 102 of the system 100 shown in FIGS. 1A-1D can be trained in an unsupervised manner using ground truth data including T cell receptor sequences. This can include jointly optimizing a set of forward layer weights of the forward pass sub-layer and a set of backward layer weights of the backward pass sub-layer, the set of forward layer weights and the set of backward layer weights being distinct from one another.

The sequence representation vector 50 can then be applied as input to one or more downstream task elements 160 for use in further tasks, such as TCR-epitope binding affinity prediction or epitope-specific TCR clustering.

### 4.4 Downstream Tasks

The amino acid embedding models' generalization performances are evaluated on two downstream tasks: TCR-epitope binding affinity prediction and epitope-specific TCR clustering.

### 4.4.1 TCR-Epitope Binding Affinity Prediction

Computational approaches that predict TCR-epitope binding affinity benefit rapid TCR screening for a target antigen and improve personalized immunotherapy. Recent computational studies formulated it as a binary classification problem that predicts a binding affinity score (0-1) given a pair of TCR and epitope sequences.

catELMo is evaluated based on the prediction performance of a binding affinity prediction model trained on its embedding, and compares with the state-of-the-art amino acid embeddings (further demonstrated in Section 4.5). First, different types of TCR and epitope embeddings are obtained using catELMo and the comparison methods. To measure the generalized prediction performance of binding affinity prediction models, each method's dataset was split into training (64%), validation (16%), and testing (20%) sets. Two splitting strategies established in Cai et al. (Cai M, Bang S, Zhang P, Lee H. ATM-TCR: TCR-epitope binding affinity prediction using a multi-head self-attention model. Frontiers in immunology. 2022;13), which is herein incorporated by reference in its entirety, are used: TCR split and epitope split. TCR split was designed to measure the models' prediction performance on out-of-sample TCRs where no TCRs in the testing set exist in the training and validation set. Epitope split was designed to measure the models' prediction performance on out-of-sample epitopes where no epitopes in the testing set exist in the training and validation set.

The downstream model architecture is the same across all embedding methods, having three linear layers where the last layer returns a binding affinity score (FIG. 1B). Taking catELMo as an example, a catELMo representation of length 1,024 is first obtained for each sequence. The TCR representation is then fed to a linear layer with 2,048 neurons, followed by a Sigmoid Linear Units (SiLU) activation function, batch normalization, and 0.3 rate dropout. Similarly, the epitope representation is fed to another linear layer with 2,048 neurons, followed by the same layers. The outputs of TCR and epitope layers are then concatenated (4,096 neurons) and passed into a linear layer with 1,024 neurons, followed by a SiLU activation function, batch normalization, and 0.3 rate dropout. Finally, the last linear layer is appended with a neuron followed by a sigmoid activation function to obtain the binding affinity score ranging from 0 to 1. The models are trained to minimize a binary cross-entropy loss via Adam optimizer. The batch size is set as 32 and the learning rate is set as 0.001. The training is stopped if either the validation loss does not decrease for 30 consecutive epochs or it iterates over 200 epochs. Finally, AUC scores of binding affinity prediction models of the different embedding methods are compared and reported.

### 4.4.2 Epitope-Specific TCR Clustering

Clustering TCRs is the first and fundamental step in TCR repertoire analysis as it can potentially identify TCR clonotypes that are condition-specific. Hierarchical clustering is applied to outputs of catELMo and the state-of-the-art amino acid embeddings (further demonstrated in Section 4.5). Clusters are also obtained from the existing TCR clustering approaches (TCRdist and GIANA). Both methods are developed on the BLOSUM62 matrix and apply nearest neighbor search to cluster TCR sequences. GIANA used the CDR3 of TCRβ chain and V gene, while TCRdist predominantly experimented with CDR1, CDR2, and CDR3 from both TCRα and TCRβ chains. The identified clusters of each method are evaluated against the ground-truth TCR groups labeled by their binding epitopes. For fair comparisons, GIANA and TCRdist are performed only on CDR3β chains with hierarchical clustering instead of the nearest neighbor search.

Different types of TCR embeddings are first obtained from catELMo and the comparison methods. All embedding methods except BLOSUM62 yield the same size representation vectors regardless of TCR length. For BLOSUM62 embedding, the sequences are padded so that all sequences are mapped to the same size vectors (further demonstrated in Section 4.5). Hierarchical clustering is then performed on TCR embeddings of each method. In detail, the clustering algorithm starts with each TCR as a cluster with size 1. It repeatedly merges the closest two clusters based on the Euclidean distance between TCR embeddings until it reaches the target number of clusters.

The normalized mutual information (NMI) is compared between the identified cluster and the ground-truth. NMI is a harmonic mean between homogeneity and completeness. Homogeneity measures how many TCRs in a cluster bind to the same epitope, while completeness measures how many TCRs binding to the same epitope are clustered together. A higher value indicates a better clustering result. It ranges from zero to one where zero indicates no mutual information found between the identified clusters and the ground-truth clusters and one indicates a perfect correlation.

### 4.5 Comparison Studies

This section demonstrates how existing amino acid embedding methods are implemented to compare with catELMo for the two TCR-related downstream tasks.

BLOSUM62. Among various types of BLOSUM matrices, BLOSUM62 is selected for comparison as it has been widely used in many TCR-related models. Embeddings are obtained by mapping each amino acid to a vector of length 24 via BLOSUM62 matrix. Since TCRs (or epitopes) have varied lengths of the sequences, each sequence is padded using IMGT method. If a TCR sequence is shorter than the predefined length 20 (or 22 for epitopes), zero-padding is added to the middle of the sequence. Otherwise, amino acids are removed from the middle of the sequence until it reaches the target length. For each TCR, 20 amino acid embedding vectors of length 24 are flattened into a vector of length 480. For each epitope, 22 amino acid embedding vectors of length 24 are flattened into a vector of length 528.

Yang et al. The 3-mer model is selected with a window size of 5 to embed TCR and epitope sequences, which is the best combination obtained from a grid search. Each 3-mer is embedded as a numeric vector of length 64. The vectors are averaged to represent a whole sequence, resulting in a vector of length 64.

SeqVec and ProtBert. Each amino acid is embedded as a numeric vector of length 1,024. The vectors are element-wisely averaged to represent a whole sequence, resulting in a vector of length 1,024.

TCRBert. Each amino acid is embedded as a numeric vector of length 768. The vectors are element-wisely averaged to represent a whole sequence with a vector of length 768.

### 4.6 Ablation Studies

Details of the experimental design and ablation studies are provided here.

4.6.1 Depth of catELMo

The effect of various depths of catELMo on TCR-epitope binding affinity prediction performance is investigated. catELMo is compared with different numbers of BILSTM layers, specifically catELMo-Shallow, catELMo, and catELMo-Deep with 2, 4 and 8 layers respectively. Other hyperparameters and the training strategy remained the same as described in Section 4.3. For each amino acid residue, the output vectors of CharCNN and four (or two, eight) BILSTM, are averaged resulting in a numerical vector of length 1,024, and then element-wise averaging is applied over all amino acids' representations to represent a whole sequence, resulting in a numerical vector of length 1,024. Embeddings from various depths are used to train binding affinity prediction models, resulting in three sets of downstream models. All settings of the downstream models remain the same as described in Section 4.4.1. The downstream models' prediction performance is compared to investigate the optimal depth of catELMo.

4.6.2 Neural Architecture of catELMo

catELMo is compared with BERT-based amino acid embedding models using another context-aware architecture, Transformer, which has shown outstanding performance in natural language processing tasks. Different sizes of BERT, a widely used Transformer-based model, are trained for amino acid embedding, named BERT-Base-TCR, BERT-Tiny-TCR, and BERT-Large-TCR. Each model has 2, 12, and 30 Transformer layers and returns 768, 768, and 1024 sizes of embeddings for each amino acid token. Their objectives, however, are focused on masked language prediction and do not include the next sentence prediction. For each TCR sequence, 15% of amino acid tokens are masked out and the model is trained to recover the masked tokens based on the remaining ones. The models are trained on the same training set as catELMo for 10 epochs. Other parameter settings are the same as TCRBert, which is included as one of the comparison models. All other settings remain the same as described in Section 4.4.1. TCRBert and BERT-Base-TCR share the same architecture, whereas TCRBert is trained on fewer training samples (PIRD). The embedding of a whole TCR sequence is obtained by average pooling over all amino acid representations. Embeddings from each model are used to train binding affinity prediction models, resulting in three sets of downstream models. The prediction performance of the downstream prediction models is compared to evaluate the architecture of catELMo.

### 4.6.3 Size of Downstream Data

This section investigates how much downstream data catELMo can save in training a binding affinity prediction model while achieving the same performance with a model trained on a full size of data. The same model is trained on different portions of the catELMo embedding dataset. In detail, k % of binding and k % of non-binding TCR-epitope pairs are selected from training (and validation) data (k=1,2, . . . , 10,20, . . . , 100), obtain catELMo embeddings for those, and are fed to train TCR-epitope binding affinity prediction models. Note that the TCR-epitope binding affinity prediction models in this experiment differ only in the number of training and validation pairs, meaning that the same testing set is used for different ks. Experiments are run ten times for each k and their average and standard deviation of AUC, recall, precision, and F1 scores are reported. Their performance is compared to those trained on a full size of the other embedding datasets. For a more detailed investigation, the same experiment is also performed on BLOSUM62 embeddings and compare it with embeddings obtained using catELMo.

### 5. Computer-Implemented System

### 5.1 Computing Device

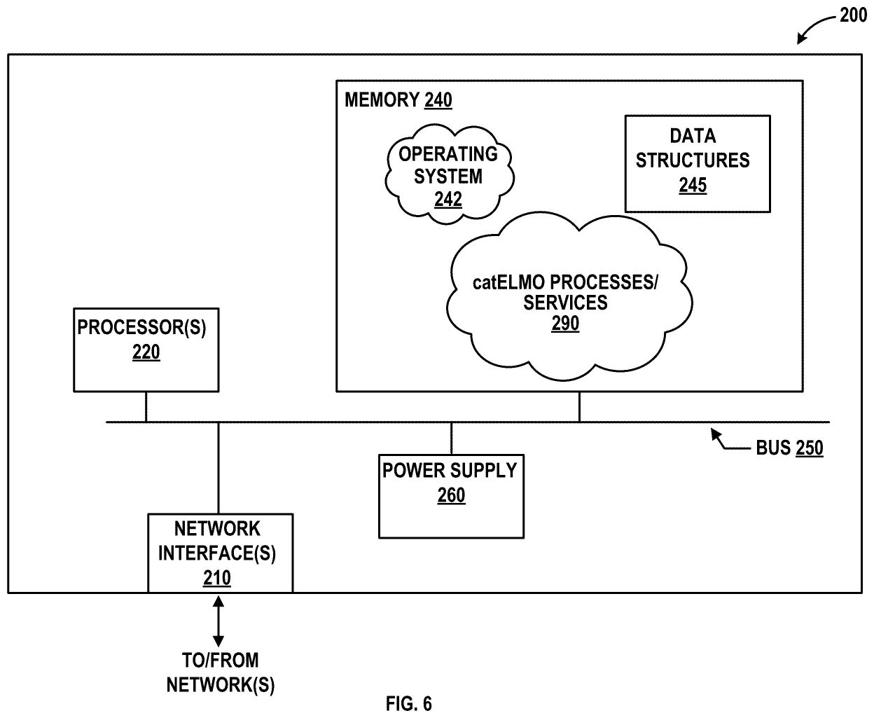

FIG. 6 is a schematic block diagram of an example device 200 that may be used with one or more embodiments described herein, e.g., implementing the system 100 of FIGS. 1A-1D including the embedding model 102 of FIGS. 1A and 1B.

Device 200 comprises one or more network interfaces 210 (e.g., wired, wireless, PLC, etc.), at least one processor 220, and a memory 240 interconnected by a system bus 250, as well as a power supply 260 (e.g., battery, plug-in, etc.).

Network interface(s) 210 include the mechanical, electrical, and signaling circuitry for communicating data over the communication links coupled to a communication network. Network interfaces 210 are configured to transmit and/or receive data using a variety of different communication protocols. As illustrated, the box representing network interfaces 210 is shown for simplicity, and it is appreciated that such interfaces may represent different types of network connections such as wireless and wired (physical) connections. Network interfaces 210 are shown separately from power supply 260, however it is appreciated that the interfaces that support PLC protocols may communicate through power supply 260 and/or may be an integral component coupled to power supply 260.

Memory 240 includes a plurality of storage locations that are addressable by processor 220 and network interfaces 210 for storing software programs and data structures associated with the embodiments described herein. In some embodiments, device 200 may have limited memory or no memory (e.g., no memory for storage other than for programs/processes operating on the device and associated caches). Memory 240 can include instructions executable by the processor 220 that, when executed by the processor 220, cause the processor 220 to implement aspects of the embedding model and associated methods outlined herein.

Processor 220 comprises hardware elements or logic adapted to execute the software programs (e.g., instructions) and manipulate data structures 245. An operating system 242, portions of which are typically resident in memory 240 and executed by the processor, functionally organizes device 200 by, inter alia, invoking operations in support of software processes and/or services executing on the device. These software processes and/or services may include catELMo processes/services 290, which can include aspects of the methods and/or implementations of various modules described herein. Note that while catELMo processes/services 290 is illustrated in centralized memory 240, alternative embodiments provide for the process to be operated within the network interfaces 210, such as a component of a MAC layer, and/or as part of a distributed computing network environment.

It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules or engines configured to operate in accordance with the techniques herein (e.g., according to the functionality of a similar process). In this context, the term module and engine may be interchangeable. In general, the term module or engine refers to model or an organization of interrelated software components/functions. Further, while the catELMo processes/services 290 is shown as a standalone process, those skilled in the art will appreciate that this process may be executed as a routine or module within other processes.

5.2 catELMo as a Computer-Implemented Process

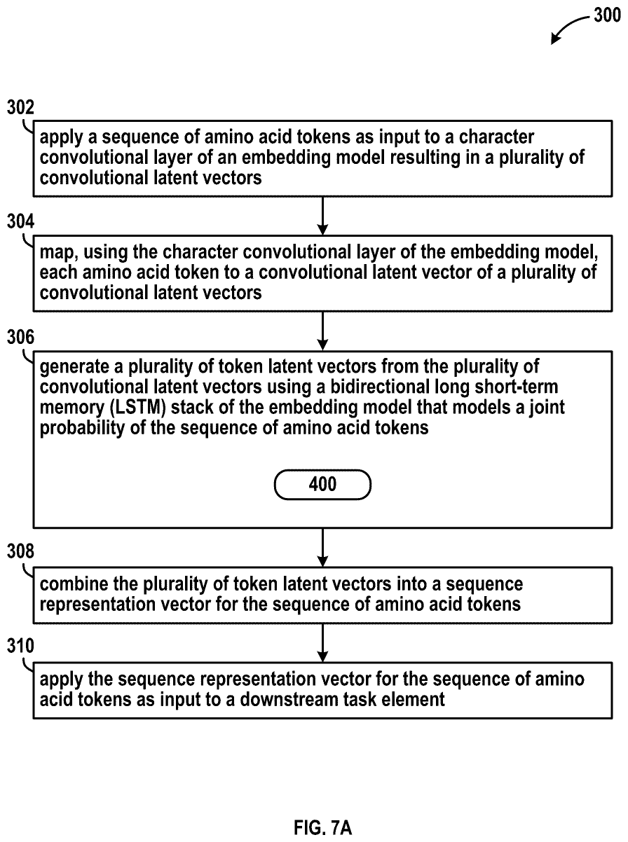

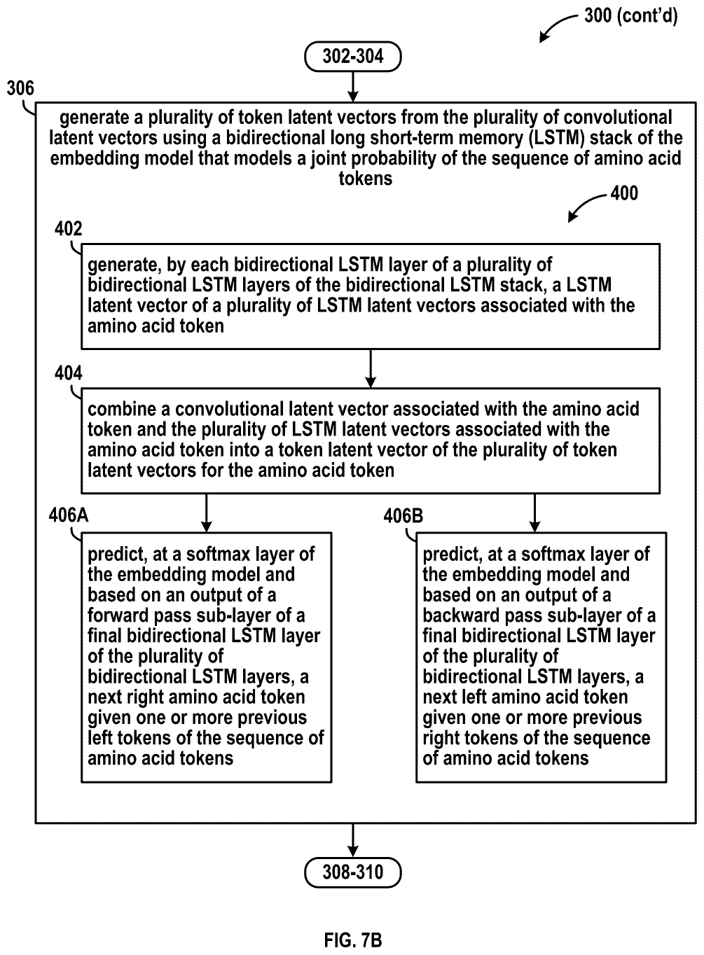

FIGS. 7A and 7B show a method 300 for generating context-aware TCR embeddings from a sequence of amino acid tokens. Method 300 discusses aspects of system 100 outlined in section 4.3 herein and shown in FIGS. 1A-1D, and may be implemented as part of catELMo processes/services 290 shown in FIG. 6.

Referring to FIG. 7A, step 302 of method 300 includes applying a sequence of amino acid tokens as input to a character convolutional layer of an embedding model resulting in a plurality of convolutional latent vectors. Step 304 of method 300 includes mapping, using the character convolutional layer of the embedding model, each amino acid token to a convolutional latent vector of a plurality of convolutional latent vectors. Each convolutional latent vector of the plurality of convolutional latent vectors is respectively associated with an amino acid token of the sequence of amino acid tokens. The amino acid token can be one-hot encoded and the convolutional latent vector can be a continuous representation vector.

Step 306 of method 300 includes generating a plurality of token latent vectors from the plurality of convolutional latent vectors using a bidirectional long short-term memory (LSTM) stack of the embedding model that models a joint probability of the sequence of amino acid tokens, each token latent vector of the plurality of token latent vectors representing an amino acid token of the sequence of amino acid tokens and encoding contextual relationships between amino acid tokens of the sequence of amino acid tokens. The bidirectional LSTM stack can have a plurality of bidirectional LSTM layers. Step 306 can include a sub-method 400, illustrated in FIG. 7B and outlined further herein.

Step 308 of method 300 includes combining the plurality of token latent vectors into a sequence representation vector for the sequence of amino acid tokens. Step 310 of method 300 includes applying the sequence representation vector for the sequence of amino acid tokens as input to a downstream task element.

Referring to FIG. 7B, step 306 can include sub-method 400. Step 402 of sub-method 400 can include generating, by each bidirectional LSTM layer of a plurality of bidirectional LSTM layers of the bidirectional LSTM stack, a LSTM latent vector of a plurality of LSTM latent vectors associated with the amino acid token. Step 404 of sub-method 400 can include combining a convolutional latent vector associated with the amino acid token and the plurality of LSTM latent vectors associated with the amino acid token into a token latent vector of the plurality of token latent vectors for the amino acid token.

Steps 406A and 406B of sub-method 400 respectively pertain to the forward and backward passes, and may be performed simultaneously. Step 406A includes predicting, at a softmax layer of the embedding model and based on an output of a forward pass sub-layer of a final bidirectional LSTM layer of the plurality of bidirectional LSTM layers, a next right amino acid token given one or more previous left tokens of the sequence of amino acid tokens. Step 406A includes predicting, at the softmax layer of the embedding model and based on an output of a backward pass sub-layer of a final bidirectional LSTM layer of the plurality of bidirectional LSTM layers, a next left amino acid token given one or more previous right tokens of the sequence of amino acid tokens.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

SEQUENCE LISTING

| SEQ ID NO. | Sequence |
|---|---|
| SEQ. ID. NO.: 1 | CASGGTGGANTGQLYF |
| SEQ. ID. NO.: 2 | MIELSLIDFYLCFLAFLLFLVLIML |
| SEQ. ID. NO.: 3 | GILGFVTFL |
| SEQ. ID. NO.: 4 | LLWNGPMAV |
| SEQ. ID. NO.: 5 | LSPRWYFYYL |
| SEQ. ID. NO.: 6 | VQELYSPIFLIV |
| SEQ. ID. NO.: 7 | GTSGSPIVNR |
| SEQ. ID. NO.: 8 | CASSLNEQF |
| SEQ. ID. NO.: 9 | CASSPTSGGQETQYF |

1. A system, comprising:
a processor in communication with a memory, the memory including instructions executable by the processor to:
apply a sequence of amino acid tokens as input to a character convolutional layer of an embedding model resulting in a plurality of convolutional latent vectors, each convolutional latent vector of the plurality of convolutional latent vectors being respectively associated with an amino acid token of the sequence of amino acid tokens;
generate a plurality of token latent vectors from the plurality of convolutional latent vectors using a bidirectional long short-term memory (LSTM) stack of the embedding model, the bidirectional LSTM stack having a plurality of bidirectional LSTM layers that collectively model a joint probability of the sequence of amino acid tokens, each token latent vector of the plurality of token latent vectors representing an amino acid token of the sequence of amino acid tokens and encoding contextual relationships between amino acid tokens of the sequence of amino acid tokens; and
combine the plurality of token latent vectors into a sequence representation vector for the sequence of amino acid tokens.

2. The system of claim 1, the bidirectional LSTM stack including a forward pass sub-layer and a backward pass sub-layer for each respective bidirectional LSTM layer of the plurality of bidirectional LSTM layers.

3. The system of claim 2, each forward pass sub-layer modeling a forward probability of a next right amino acid token of the sequence of amino acid tokens given one or more previous left tokens of the sequence of amino acid tokens.

4. The system of claim 1, the memory further including instructions executable by the processor to:
predict, at a softmax layer of the embedding model and based on an output of a forward pass sub-layer of a final bidirectional LSTM layer of the plurality of bidirectional LSTM layers, a next right amino acid token given one or more previous left tokens of the sequence of amino acid tokens.

5. The system of claim 2, each backward pass sub-layer modeling a backward probability of a next left amino acid token of the sequence of amino acid tokens given one or more previous right tokens of the sequence of amino acid tokens.

6. The system of claim 1, the memory further including instructions executable by the processor to:
predict, at a softmax layer of the embedding model and based on an output of a backward pass sub-layer of a final bidirectional LSTM layer of the plurality of bidirectional LSTM layers, a next left amino acid token given one or more previous right tokens of the sequence of amino acid tokens.

7. The system of claim 2, the forward pass sub-layer having a set of forward layer weights and the backward pass sub-layer having a set of backward layer weights that are jointly optimized during a training process of the embedding model, the set of forward layer weights and the set of backward layer weights being distinct from one another.

8. The system of claim 1, each bidirectional LSTM layer of the plurality of bidirectional LSTM layers respectively outputting a LSTM latent vector of a plurality of LSTM latent vectors associated with the amino acid token, the memory further including instructions executable by the processor to:
combine a convolutional latent vector associated with the amino acid token and the plurality of LSTM latent vectors associated with the amino acid token into a token latent vector of the plurality of token latent vectors for the amino acid token.

9. The system of claim 1, the sequence representation vector for the sequence of amino acid tokens being an element-wise average of the plurality of token latent vectors.

10. The system of claim 1, the character convolutional layer including a plurality of convolutional layers, each convolutional layer of the plurality of convolutional layers being followed by a maxpooling layer, the memory further including instructions executable by the processor to:
map, using the character convolutional layer of the embedding model, each amino acid token to a convolutional latent vector of the plurality of convolutional latent vectors, the amino acid token being one-hot encoded and the convolutional latent vector being a continuous representation vector.

11. The system of claim 1, the embedding model having been trained using ground truth data including T cell receptor sequences.

12. The system of claim 1, the memory further including instructions executable by the processor to:
train the embedding model in an unsupervised manner using ground truth data including T cell receptor sequences.

13. The system of claim 1, the memory further including instructions executable by the processor to:
apply the sequence representation vector for the sequence of amino acid tokens as input to a downstream task element.

14. A method, comprising:
applying a sequence of amino acid tokens as input to a character convolutional layer of an embedding model resulting in a plurality of convolutional latent vectors, each convolutional latent vector of the plurality of convolutional latent vectors being respectively associated with an amino acid token of the sequence of amino acid tokens;
generating a plurality of token latent vectors from the plurality of convolutional latent vectors using a bidirectional long short-term memory (LSTM) stack of the embedding model, the bidirectional LSTM stack having a plurality of bidirectional LSTM layers that collectively model a joint probability of the sequence of amino acid tokens, each token latent vector of the plurality of token latent vectors representing an amino acid token of the sequence of amino acid tokens and encoding contextual relationships between amino acid tokens of the sequence of amino acid tokens; and
combining the plurality of token latent vectors into a sequence representation vector for the sequence of amino acid tokens.

15. The method of claim 14, further comprising:
predicting, at a softmax layer of the embedding model and based on an output of a forward pass sub-layer of a final bidirectional LSTM layer of the plurality of bidirectional LSTM layers, a next right amino acid token given one or more previous left tokens of the sequence of amino acid tokens; and
predicting, at the softmax layer of the embedding model and based on an output of a backward pass sub-layer of a final bidirectional LSTM layer of the plurality of bidirectional LSTM layers, a next left amino acid token given one or more previous right tokens of the sequence of amino acid tokens.

16. The method of claim 15, further comprising:
jointly optimizing a set of forward layer weights of the forward pass sub-layer and a set of backward layer weights of the backward pass sub-layer, the set of forward layer weights and the set of backward layer weights being distinct from one another.

17. The method of claim 14, the embedding model having been trained using ground truth data including T cell receptor sequences.

18. The method of claim 14, further comprising:
training the embedding model in an unsupervised manner using ground truth data including T cell receptor sequences.

19. The method of claim 14, further comprising:
applying the sequence representation vector for the sequence of amino acid tokens as input to a downstream task element.

20. A non-transitory computer readable medium including instructions encoded thereon that are executable by a processor to:
apply a sequence of amino acid tokens as input to a character convolutional layer of an embedding model resulting in a plurality of convolutional latent vectors, each convolutional latent vector of the plurality of convolutional latent vectors being respectively associated with an amino acid token of the sequence of amino acid tokens;
generate a plurality of token latent vectors from the plurality of convolutional latent vectors using a bidirectional long short-term memory (LSTM) stack of the embedding model, the bidirectional LSTM stack having a plurality of bidirectional LSTM layers that collectively model a joint probability of the sequence of amino acid tokens, each token latent vector of the plurality of token latent vectors representing an amino acid token of the sequence of amino acid tokens and encoding contextual relationships between amino acid tokens of the sequence of amino acid tokens; and
combine the plurality of token latent vectors into a sequence representation vector for the sequence of amino acid tokens.
